(19) United States
(12) Patent Application Publication
(10) Pub. No.: US 2014/0378474 A1
(43) Pub. Date: Dec. 25, 2014

(54) 5-membered heteroarylcarboxamide derivatives as plasma kallikrein inhibitors

(71) Applicant: NOVARTIS AG, Basel (CH)

(72) Inventors: Stefanie Flohr, Lorrach (DE); Christian Markert, Riehen (CH); Kenji Namoto, Basel (CH); Bernard Pirard, Hegenheim (FR)

(73) Assignee: NOVARTIS AG, Basel (CH)

(21) Appl. No.: 14/371,457

(22) PCT Filed: Jan. 25, 2013
(86) PCT No.: PCT/IB2013/050662

§ 371 Date: Jul. 10, 2014

Related U.S. Application Data
(60) Provisional application No. 61/591,472, filed on Jan. 27, 2012.

(51) Int. Cl. C07D 471/04; C07D 401/14; A61K 31/4155; C07D 403/10; C07D 231/14; C07D 413/14; A61K 31/497; C07D 401/06; A61K 31/4439; C07D 403/14; A61K 31/422; A61K 31/4709; C07D 403/12

(52) U.S. Cl. CPC C07D 471/04; A61K 31/4709; C07D 401/14; A61K 31/4155; C07D 403/10; C07D 231/14; C07D 403/12; A61K 31/497; C07D 401/06; A61K 31/4439; C07D 403/14; A61K 31/422; C07D 413/14
USPC 514/255.05; 514/314; 546/166; 514/300; 546/113; 514/406; 548/365.1; 548/374.1; 548/362.5; 544/405; 514/338; 546/275.7; 514/374; 548/229

(57) ABSTRACT

The invention relates to a compound of the formula (I) in which the substituents are as defined in the specification; in free form or in salt form; to its preparation, to its use as medicament and to medicaments comprising it.

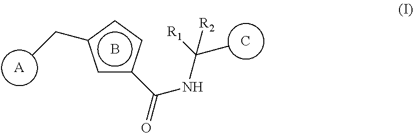

The invention relates to 5-membered heteroarylcarboxamide derivatives, to their preparation, to their use as medicaments and to medicaments comprising them.

Plasmakallikrein (PK) is the activated form of the trypsin-like serine protease plasma-prokallikrein and is mainly expressed by hepatocytes in the liver. Activation of plasma-prokallikrein is believed to be mainly mediated through coagulation factor XIIa (fXIIa). Binding of the zymogen factor XII (fXII) to negatively charged surfaces is thought to induce a major conformational change in the protein, resulting in the expression of endogeneous (auto)activity sufficient to activate a small number of plasma-prokallikrein molecules. In a positive feedback mechanism, active plasmakallikrein efficiently activates surface-bound fXII to fXIIa and vice versa. This reciprocal activation of fXII and plasmakallikrein is critical for the formation of sufficient plasmakallikrein activity to trigger downstream proteolytic cascades. FXIIa is the first component of the intrinsic pathway of coagulation activating factor XI to factor XIa. Moreover, plasmakallikrein activated by fXIIa cleaves high molecular weight kininogen to bradykinin (BK). The nonapeptide BK is a potent mediator of inflammation, vasodilation, pain and increased vascular permeability. The functional C1 esterase inhibitor (C1Inh) regulates the activation of several proteolytic systems in plasma and is the major endogeneous inhibitor of PK. Low molecular weight plasmakallikrein inhibitors are described e.g. in WO03/076458, WO2008016883.

Plasma kallikrein may have numerous implications in disorders such as hereditary angioedema (HAE) (J A Bernstein et al, Expert Rev. Clin. Immunol., 6, 29-39, 2010; UC Nzeako et al., Arch Intern Med., 161, 2417-2429, 2001), retinopathy or diabetic retinopathy (A C Clermont et al, Abstract 5035-D883, ARVO 2010, Fort Lauderdale, Fla.), proliferative and non-proliferative retinopathy, diabetic macular edema (DME), clinically significant macular edema (CSME), cystoid macular edema (CME), CME following cataract extraction, CME induced by cryotherapy, CME induced by uveitis, CME following vascular occlusion (e.g. central retina vein occlusion, branch retinal vein occlusion, or hemiretinal vein occlusion), retinal edema, complications related to cataract surgery in diabetic retinopathy, hypertensive retinopathy (J A Phipps et al, Hypertension, 53, 175-181, 2009), retinal trauma, dry and wet aged-related macular degeneration (AMD), ischemic reperfusion injuries (C Storoni et al, JPET, 318, 849-954, 2006), e.g. in all kind of contexts associated with tissue and/or organ transplantation, surgically-induced brain injury, focal cerebral ischemia, global cerebral ischemia, glioma-associated edema, spinal cord injury, pain, ischemia, focal brain ischemia, neurological and cognitive deficits, deep vein thrombosis, stroke, myocardial infarction, acquired angioedema drug-related (ACE-inhibitors), edema, high altitude cerebral edema, cytotoxic cerebral edema, osmotic cerebral edema, obstructive hydrocephalus, radiation induced edema, lymph edema, traumatic brain injury, hemorrhagic stroke (e.g., cerebral stroke or subarachnoid stroke), intracerebral hemorrhage, hemorrhagic transformation of ischemic stroke, cerebral trauma associate with injury or surgery, brain aneurysm, arterio-venous malformation, reduction of blood losses during surgical procedures (e.g. cardiothoracic surgery, such as cardiopulmonary bypass or coronary artery bypass grafting), blood coagulation disorders such as thrombosis, itch, disorders with an inflammation component (such as multiple sclerosis), epilepsy, encephalitis, Alzheimer's disease, excessive daytime sleepiness, essential hypertension, increased blood pressure associated with diabetes or hyperlipidemia, renal insufficiency, chronic kidney disease, heart failure, microalbuminuria, albuminuria, proteinuria, disorders associated with increased vascular permeability (e.g. increased retinal vascular permeability, increased leg, feet, ankle vascular permeability), cerebral hemorrhage, microalbuminuria, albuminuria and proteinuria, deep vein thrombosis, coagulation from post fibrinolytic treatments, angina, angioedema, sepsis, arthritis (e.g. rheumatoid arthritis, osteoarthritis, infection arthritis), lupus, gout, psoriasis, blood loss during cardiopulmonary bypass, inflammatory bowel, diabetes, diabetic complications, infectious diseases, astrocyte-activation related diseases (e.g. Alzheimer's disease or multiple sclerosis), Parkinson's disease, amyotrophic lateral sclerosis, Creutzfeld-Jacob disease, stroke, epilepsy and trauma (e.g. brain trauma), allergic edema e.g. airflow obstruction in chronic allergic sinusitis or perennial rhinitis; airflow obstruction in acute asthma; serositis associated with systemic lupus erythematosus (SLE) and other diseases.

Plasma kallikrein inhibitors are considered to be useful in the treatment of a wide range of disorders, in particular retinopathy or edema-associated diseases, such as hereditary angioedema, macular edema and brain edema.

Plasma kallikrein inhibitors are considered to be especially useful in the treatment of retinopathy, e.g. retinopathy associated with diabetes and/or hypertension.

Plasma kallikrein inhibitors are considered to be especially useful in the treatment of hereditary angioedema.

Plasma kallikrein inhibitors are considered to be especially useful in the treatment of edema formation in diseases, e.g. edema formation related to ischemic reperfusion injuries.

Plasma kallikrein inhibitors are considered to be especially useful in the treatment of macular edema, e.g. macular edema associated with diabetes and/or hypertension.

There is a need to provide new plasmakallikrein inhibitors that are good drug candidates. In particular, preferred compounds should bind potently to plasmakallikrein whilst showing little affinity for other proteases. They should be well absorbed from the gastrointestinal tract, be sufficiently metabolically stable and possess favorable pharmacokinetic properties. They should be non-toxic and demonstrate few side-effects. Furthermore, the ideal drug candidate will be able to exist in a physical form that is stable, non-hygroscopic and easily formulated.

The compounds of the invention are plasmakallikrein inhibitors and are therefore potentially useful in the treatment of a wide range of disorders, particularly retinopathy or edema-associated diseases.

In a first aspect, the invention relates to a compound of the formula I in free form or in pharmaceutically acceptable salt form

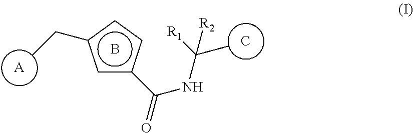

wherein

R₁ and R₂ are each independently hydrogen or methyl;

A is a 5- to 10-membered monocyclic or fused polycyclic aromatic ring system, which may contain 1, 2, 3, or 4 heteroatoms selected from N, O and S, wherein the ring system

A is unsubstituted or substituted once, twice or three times by R₃;

wherein A is neither unsubstituted phenyl nor unsubstituted pyridinyl;

each R₃ is independently selected from halogen, C₁-C₄alkyl, C₁-C₄alkoxy, oxo, cyano, C₁-C₄halogenalkyl, NR₄R₅; or

R₃ is a 5- to 6-membered monocyclic ring system which may be aromatic, saturated or unsaturated non-aromatic and which may contain 1, 2, 3 or 4 heteroatoms selected from N, O and S, wherein the ring system R₃ is attached to A via bond, C₁-C₂alkylene or SO₂,

wherein the ring system R₃ is in turn optionally substituted with oxo;

R₄ and R₅ are independently selected from hydrogen or C₁-C₄alkyl;

B is a five-membered monocyclic aromatic ring system which contains 1, 2, 3, or 4 heteroatoms selected from N, O and S;

C is a 5- to 10-membered monocyclic or fused polycyclic aromatic ring system, which may contain 1, 2, 3, or 4 heteroatoms selected from N, O and S, wherein the ring system C is unsubstituted or substituted once, twice or three times by R₆;

each R₆ is independently selected from halogen, C₁-C₄alkyl, C₁-C₄alkoxy, C₁-C₄halogenalkyl, amino, aminoC₁-C₄alkyl, cyano, C₂-C₄alkynyl;

wherein C is neither a 2-aminopyridinyl nor a 6-aminopyridinyl residue,

for use as a medicament.

Unless specified otherwise, the term “compounds of the present invention” refers to compounds of formula (I), salts of the compound, hydrates or solvates of the compounds, salts, as well as all stereoisomers (including diastereoisomers and enantiomers), tautomers and isotopically labeled compounds (including deuterium substitutions), as well as inherently formed moieties (e.g. polymorphs, solvates and/or hydrates).

For purposes of interpreting this specification, the following definitions will apply and whenever appropriate, terms used in the singular will also include the plural and vice versa.

As used herein, “alkyl” represents a straight-chain or branched-chain alkyl group, for example, methyl, ethyl, n- or iso-propyl, n-, iso-, sec- or tert-butyl; C₁₋₄alkyl preferably represents a straight-chain or branched-chain C₁₋₄alkyl with particular preference given to methyl, ethyl, n-propyl, iso-propyl and tert-butyl.

Each alkyl part of “alkoxy”, “halogenalkyl” and so on shall have the same meaning as described in the above-mentioned definition of “alkyl”, especially regarding linearity and preferential size.

As used herein, the term “halogen” or “halo” refers to fluoro, chloro, bromo, and iodo, preferably fluoro or chloro.

Halogenalkyl groups preferably have a chain length of 1 to 4 carbon atoms and are, for example, fluoromethyl, difluoromethyl, trifluoromethyl, chloromethyl, dichloromethyl, trichloromethyl, 2,2,2-trifluoroethyl, 2-fluoroethyl, 2-chloroethyl, pentafluoroethyl, 1,1-difluoro-2,2,2-trichloroethyl, 2,2,2-trichloroethyl, 1,1,2,2-tetrafluoroethyl, 2,2,3,3-tetrafluoropropyl, 2,2,3,3,3-pentafluoropropyl or 2,2,3,4,4,4-hexafluorobutyl; preferably —CF₃, —CHF₂, —CH₂F, —CHF—CH₃, CF₂CH₃, or CH₂CF₃.

As used herein, the term “C₁₋₄alkoxy” refers to C₁₋₄alkyl-O—, wherein C₁₋₄alkyl is defined herein above. Representative examples of C₁₋₄alkoxy include, but are not limited to, methoxy, ethoxy, propoxy, 2-propoxy, butoxy, tert-butoxy.

As used herein, the term “C₁-C₂alkylene” refers to divalent alkyl group as defined herein above having 1 to 2 carbon atoms, such as methylene or ethylene.

In the context of the invention, the definition of A or C as a “5- to 10-membered monocyclic or fused polycyclic aromatic ring system which may contain 1, 2, 3 or 4 heteroatoms” encompasses a C₆- or C₁₀-aromatic hydrocarbon group. It also encompasses a five-, six-, nine- or ten-membered heterocyclic aromatic ring system.

A C₆- or C₁₀-aromatic hydrocarbon group is typically phenyl or naphthyl respectively. A C₆-aromatic hydrocarbon group is especially phenyl.

Examples of heterocyclic aromatic ring systems are: imidazo[2,1-b]thiazole, pyrrole, pyrazole, imidazole, triazole, tetrazole, furane, oxadiazole, thiophene, oxazole, isoxazole, thiazole, isothiazole, thiadiazole, pyridine, pyridazine, pyrazine, triazine, oxazine, thiazine, dioxine, purine, pteridine, and the corresponding benz-annelated heterocycles, e.g. indole, isoindole, coumarin, isoquinoline, quinoline and the like.

The term “fused polycyclic aromatic ring system” refers to an aromatic substituent which consists of multiple, e.g. two aromatic rings that are fused together.

“Polycyclic” means preferably bicyclic.

In the context of the invention, the definition of R₃ as a “5- to 6-membered monocyclic ring system which may be aromatic, saturated or unsaturated non-aromatic and which may contain from 1, 2, 3, or 4 heteroatoms” encompasses 5- to 6-membered monocyclic aromatic or non-aromatic hydrocarbon groups and aromatic or non-aromatic heterocyclic ring systems of the same sizes. Typically, it encompasses a six-membered monocyclic aromatic hydrocarbon group, a six-membered monocyclic heterocyclic aromatic ring system, a six-membered non-aromatic saturated or unsaturated monocyclic hydrocarbon group, a six-membered non-aromatic saturated or unsaturated monocyclic heterocycle, a five-membered aromatic monocyclic heterocycle, a five-membered non-aromatic heterocycle.

In the context of the invention, the definition of B as a “five-membered monocyclic aromatic ring system which contains 1, 2, 3, or 4 heteroatoms selected from N, O and S” preferably includes ring system comprising 5 ring atoms of which 1, 2 or 3 ring atoms are heteroatoms. Typically, B is a pyrrole, pyrazole or triazole.

Various enumerated embodiments of the invention are described herein. It will be recognized that features specified in each embodiment may be combined with other specified features to provide further embodiments of the present invention.

In one embodiment, the invention provides a compound of the formula (I), or a salt thereof, as described above.

In one embodiment, the invention provides a compound of formula (I) in free form or in pharmaceutically acceptable salt form wherein

R₁ and R₂ are both hydrogen;

A is a 9- or 10-membered fused bicyclic aromatic ring system which may contain 1, 2, 3, or 4 hetereoatoms selected from N, O and S, wherein the ring system A is unsubstituted;

B is a five-membered monocyclic aromatic ring system which contains 1, 2, 3 or 4 nitrogen atoms;

C is a 5- to 10-membered monocyclic or bicyclic aromatic ring system, which may contain 1, 2, 3, or 4 heteroatoms selected from N, O and S, wherein the ring system C is substituted once, twice or three times by R₆;

wherein each R₆ is independently selected from halogen, C₁-C₄alkyl, C₁-C₄alkoxy, C₁-C₄halogenalkyl, amino, aminoC₁-C₄alkyl, —(C═NH)NH₂, cyano, C₂-C₄alkynyl;

wherein C is neither a 2-aminopyridinyl nor a 6-aminopyridinyl residue.

In one embodiment, the invention provides a compound of formula (I) in free form or in pharmaceutically acceptable salt form wherein

R₁ and R₂ are both hydrogen;

A is naphthyl;

B is a five-membered monocyclic aromatic ring system which contains 1, 2, 3 or 4 nitrogen atoms;

C is a 5- to 10-membered monocyclic or bicyclic aromatic ring system, which may contain 1, 2, 3, or 4 heteroatoms selected from N, O and S, wherein the ring system C is substituted once, twice or three times by R₆;

wherein each R₆ is independently selected from halogen, C₁-C₄alkyl, C₁-C₄alkoxy, C₁-C₄halogenalkyl, amino, aminoC₁-C₄alkyl, —(C═NH)NH₂, cyano, C₂-C₄alkynyl;

wherein C is neither a 2-aminopyridinyl nor a 6-aminopyridinyl residue.

In one embodiment, the invention provides a compound of formula (I) in free form or in pharmaceutically acceptable salt form wherein

R₁ and R₂ are both hydrogen;

A is a 9- or 10-membered fused bicyclic aromatic ring system which contains 1, 2, 3, or 4 hetereoatoms selected from N, O and S, wherein the ring system A is substituted once by R₃;

wherein R₃ is selected from halogen, C₁-C₄alkyl, C₁-C₄alkoxy, or C₁-C₄halogenalkyl;

B is a five-membered monocyclic aromatic ring system which contains 1, 2, 3 or 4 nitrogen atoms;

C is a 5- to 10-membered monocyclic or bicyclic aromatic ring system, which may contain 1, 2, 3, or 4 heteroatoms selected from N, O and S, wherein the ring system C is substituted once, twice or three times by R₆;

wherein each R₆ is independently selected from halogen, C₁-C₄alkyl, C₁-C₄alkoxy, C₁-C₄halogenalkyl, amino, aminoC₁-C₄alkyl, —(C═NH)NH₂, cyano, C₂-C₄alkynyl;

wherein C is neither a 2-aminopyridinyl nor a 6-aminopyridinyl residue.

In one embodiment, the invention provides a compound of formula (I) in free form or in pharmaceutically acceptable salt form wherein

R₁ and R₂ are both hydrogen;

A is a 6-membered aromatic ring system which may contain 1, 2, 3, or 4 hetereoatoms selected from N, O and S, wherein the ring system A is substituted once by R₃;

wherein R₃ is a 5- to 6-membered monocyclic ring system which may be aromatic, saturated or unsaturated non-aromatic and which may contain 1, 2, 3 or 4 heteroatoms selected from N, O and S, wherein the ring system R₃ is attached to A via methylene or SO₂, wherein the ring system R₃ is in turn optionally substituted with oxo;

B is a five-membered monocyclic aromatic ring system which contains 1, 2, 3 or 4 nitrogen atoms;

C is a 5- to 10-membered monocyclic or bicyclic aromatic ring system, which may contain 1, 2, 3, or 4 heteroatoms selected from N, O and S, wherein the ring system C is substituted once, twice or three times by R₆;

wherein each R₆ is independently selected from halogen, C₁-C₄alkyl, C₁-C₄alkoxy, C₁-C₄halogenalkyl, amino, aminoC₁-C₄alkyl, —(C═NH)NH₂, cyano, C₂-C₄alkynyl;

wherein C is neither a 2-aminopyridinyl nor a 6-aminopyridinyl residue.

In one embodiment, the invention provides a compound of formula (I) in free form or in pharmaceutically acceptable salt form wherein

R₁ and R₂ are both hydrogen;

A is a 6-membered aromatic ring system which may contain 1, 2, 3, or 4 hetereoatoms selected from N, O and S, wherein the ring system A is substituted once or twice by R₃;

wherein each R₃ is independently selected from halogen, C₁-C₄alkyl, C₁-C₄alkoxy, or C₁-C₄halogenalkyl;

B is a five-membered monocyclic aromatic ring system which contains 1, 2, 3 or 4 nitrogen atoms;

C is a 5- to 10-membered monocyclic or bicyclic aromatic ring system, which may contain 1, 2, 3, or 4 heteroatoms selected from N, O and S, wherein the ring system C is substituted once, twice or three times by R₆;

wherein each R₆ is independently selected from halogen, C₁-C₄alkyl, C₁-C₄alkoxy, C₁-C₄halogenalkyl, amino, aminoC₁-C₄alkyl, cyano, C₂-C₄alkynyl;

wherein C is neither a 2-aminopyridinyl nor a 6-aminopyridinyl residue, for use as a medicament.

In one embodiment, the invention provides a compound of formula (I) in free form or in pharmaceutically acceptable salt form wherein

R₁ and R₂ are both hydrogen;

A is phenyl substituted once by R₃;

wherein R₃ is a 5- to 6-membered monocyclic ring system which may be aromatic, saturated or unsaturated non-aromatic and which may contain 1, 2, 3 or 4 heteroatoms selected from N, O and S, wherein the ring system R₃ is attached to A via methylene or SO₂, wherein the ring system R₃ is in turn optionally substituted with oxo;

B is a five-membered monocyclic aromatic ring system which contains 1, 2, 3 or 4 nitrogen atoms;

C is a 5- to 10-membered monocyclic or bicyclic aromatic ring system, which may contain 1, 2, 3, or 4 heteroatoms selected from N, O and S, wherein the ring system C is substituted once, twice or three times by R₆;

wherein each R₆ is halogen, C₁-C₄alkyl, C₁-C₄alkoxy, C₁-C₄halogenalkyl, amino, aminoC₁-C₄alkyl, —(C═NH)NH₂, cyano, C₂-C₄alkynyl;

wherein C is neither a 2-aminopyridinyl nor a 6-aminopyridinyl residue.

In one embodiment, the invention provides a compound of formula (I) in free form or in pharmaceutically acceptable salt form wherein

R₁ and R₂ are both hydrogen;

A is a 5- to 10-membered monocyclic or fused polycyclic aromatic ring system, which may contain 1, 2, 3, or 4 heteroatoms selected from N, O and S, wherein the ring system

A is unsubstituted or substituted once, twice or three times by R₃;

wherein A is neither unsubstituted phenyl nor unsubstituted pyridinyl;

each R₃ is independently selected from halogen, C₁-C₄alkyl, C₁-C₄alkoxy, oxo, cyano, C₁-C₄halogenalkyl, NR₄R₅; or

R₃ is a 5- to 6-membered monocyclic ring system which may be aromatic, saturated or unsaturated non-aromatic and which may contain 1, 2, 3 or 4 heteroatoms selected from N, O and S, wherein the ring system R₃ is attached to A via methylene or SO₂, wherein the ring system R₃ is in turn optionally substituted with oxo;

R₄ and R₅ are independently selected from hydrogen or C₁-C₄alkyl;

B is selected from

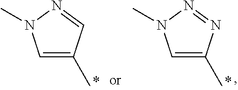

wherein the bond marked with * is attached to the carboxamide group;

C is a 5- to 10-membered monocyclic or bicyclic aromatic ring system, which may contain 1, 2, 3, or 4 heteroatoms selected from N, O and S, wherein the ring system C is substituted once, twice or three times by R₆;

wherein each R₆ is halogen, C₁-C₄alkyl, C₁-C₄alkoxy, C₁-C₄halogenalkyl, amino, aminoC₁-C₄alkyl, cyano, C₂-C₄alkynyl;

wherein C is neither a 2-aminopyridinyl nor a 6-aminopyridinyl residue, for use as a medicament.

In one embodiment, the invention provides a compound of formula (I) in free form or in pharmaceutically acceptable salt form wherein

R₁ and R₂ are both hydrogen;

A is a 5- to 10-membered monocyclic or fused polycyclic aromatic ring system, which may contain 1, 2, 3, or 4 heteroatoms selected from N, O and S, wherein the ring system

A is unsubstituted or substituted once, twice or three times by R₃;

wherein A is neither unsubstituted phenyl nor unsubstituted pyridinyl;

each R₃ is independently selected from halogen, C₁-C₄alkyl, C₁-C₄alkoxy, oxo, cyano, C₁-C₄halogenalkyl, NR₄R₅; or

R₃ is a 5- to 6-membered monocyclic ring system which may be aromatic, saturated or unsaturated non-aromatic and which may contain 1, 2, 3 or 4 heteroatoms selected from N, O and S, wherein the ring system R₃ is attached to A via methylene or SO₂, wherein the ring system R₃ is in turn optionally substituted with oxo;

R₄ and R₅ are independently selected from hydrogen or C₁-C₄alkyl;

B is selected from

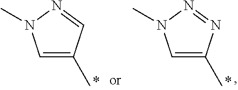

wherein the bond marked with * is attached to the carboxamide group;

C is a 5- to 10-membered monocyclic or bicyclic aromatic ring system, which may contain 1, 2, 3, or 4 heteroatoms selected from N, O and S, wherein the ring system C is substituted once by R₆;

wherein R₆ is selected from halogen, C₁-C₄alkyl, C₁-C₄alkoxy or C₁-C₄halogenalkyl, for use as a medicament.

In one embodiment, the invention provides a compound of formula (I) in free form or in pharmaceutically acceptable salt form wherein

R₁ and R₂ are both hydrogen;

A is a 5- to 10-membered monocyclic or fused polycyclic aromatic ring system, which may contain 1, 2, 3, or 4 heteroatoms selected from N, O and S, wherein the ring system

A is unsubstituted or substituted once, twice or three times by R₃;

wherein A is neither unsubstituted phenyl nor unsubstituted pyridinyl;

each R₃ is independently selected from halogen, C₁-C₄alkyl, C₁-C₄alkoxy, oxo, cyano, C₁-C₄halogenalkyl, NR₄R₅; or

R₃ is a 5- to 6-membered monocyclic ring system which may be aromatic, saturated or unsaturated non-aromatic and which may contain 1, 2, 3 or 4 heteroatoms selected from N, O and S, wherein the ring system R₃ is attached to A via methylene or SO₂, wherein the ring system R₃ is in turn optionally substituted with oxo;

R₄ and R₅ are independently selected from hydrogen or C₁-C₄alkyl;

B is selected from

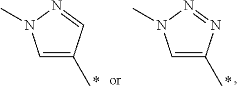

wherein the bond marked with * is attached to the carboxamide group;

C is a 9-membered bicyclic aromatic ring system, which contains 1 or 2 nitrogen atoms,

wherein the ring system C is substituted once by R₆;

wherein R₆ is selected from halogen or C₁-C₄alkyl.

In one embodiment, the invention provides a compound of formula (I) in free form or in pharmaceutically acceptable salt form wherein

R₁ and R₂ are both hydrogen;

A is a 5- to 10-membered monocyclic or fused polycyclic aromatic ring system, which may contain 1, 2, 3, or 4 heteroatoms selected from N, O and S, wherein the ring system

A is unsubstituted or substituted once, twice or three times by R₃;

wherein A is neither unsubstituted phenyl nor unsubstituted pyridinyl;

each R₃ is independently selected from halogen, C₁-C₄alkyl, C₁-C₄alkoxy, oxo, cyano, C₁-C₄halogenalkyl, NR₄R₅; or

R₃ is a 5- to 6-membered monocyclic ring system which may be aromatic, saturated or unsaturated non-aromatic and which may contain 1, 2, 3 or 4 heteroatoms selected from N, O and S, wherein the ring system R₃ is attached to A via methylene or SO₂, wherein the ring system R₃ is in turn optionally substituted with oxo;

R₄ and R₅ are independently selected from hydrogen or C₁-C₄alkyl;

B is selected from

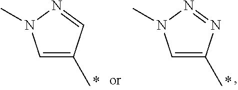

wherein the bond marked with * is attached to the carboxamide group;

C is indolyl or indazolyl substituted once by R₆;

wherein R₆ is selected from halogen or C₁-C₄alkyl.

In one embodiment, the invention provides a compound of formula (I) in free form or in pharmaceutically acceptable salt form wherein

R₁ and R₂ are both hydrogen;

A is a 5- to 10-membered monocyclic or fused polycyclic aromatic ring system, which may contain 1, 2, 3, or 4 heteroatoms selected from N, O and S, wherein the ring system

A is unsubstituted or substituted once, twice or three times by R₃;

wherein A is neither unsubstituted phenyl nor unsubstituted pyridinyl;

each R₃ is independently selected from halogen, C₁-C₄alkyl, C₁-C₄alkoxy, oxo, cyano, C₁-C₄halogenalkyl, NR₄R₅; or

R₃ is a 5- to 6-membered monocyclic ring system which may be aromatic, saturated or unsaturated non-aromatic and which may contain 1, 2, 3 or 4 heteroatoms selected from N, O and S, wherein the ring system R₃ is attached to A via methylene or SO₂, wherein the ring system R₃ is in turn optionally substituted with oxo;

R₄ and R₅ are independently selected from hydrogen or C₁-C₄alkyl;

B is selected from

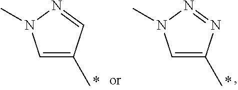

wherein the bond marked with * is attached to the carboxamide group;

C is a 6-membered aromatic ring system, which may contain 1, 2, 3 or 4 heteroatoms selected from N, O and S, wherein the ring system is substituted once by R₆;

wherein R₆ is selected from halogen, C₁-C₄alkyl, C₁-C₄alkoxy or C₁-C₄halogenalkyl, for use as a medicament.

In one embodiment, the invention provides a compound of formula (I) in free form or in pharmaceutically acceptable salt form wherein

R₁ and R₂ are both hydrogen;

A is a 6-membered aromatic ring system which may contain 1, 2, 3, or 4 hetereoatoms selected from N, O and S, wherein the ring system A is substituted once by R₃;

wherein R₃ is a 5- to 6-membered monocyclic ring system which may be aromatic, saturated or unsaturated non-aromatic and which may contain 1, 2, 3 or 4 heteroatoms selected from N, O and S, wherein the ring system R₃ is attached to A via methylene or SO₂, wherein the ring system R₃ is in turn optionally substituted with oxo;

R₄ and R₅ are independently selected from hydrogen or C₁-C₄alkyl;

B is selected from

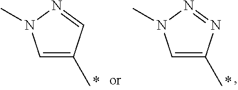

wherein the bond marked with * is attached to the carboxamide group;

C is a 9-membered bicyclic aromatic ring system, which contains 1 or 2 nitrogen atoms,

wherein the ring system C is substituted once by R₆;

wherein R₆ is selected from halogen or C₁-C₄alkyl.

In one embodiment, the invention provides a compound of formula (I) in free form or in pharmaceutically acceptable salt form wherein

R₁ and R₂ are both hydrogen;

A is a 9- or 10-membered fused bicyclic aromatic ring system which may contain 1, 2, 3, or 4 hetereoatoms selected from N, O and S, wherein the ring system A is unsubstituted or substituted once by R₃;

wherein R₃ is selected from halogen, C₁-C₄alkyl, C₁-C₄alkoxy, or C₁-C₄halogenalkyl;

R₄ and R₅ are independently selected from hydrogen or C₁-C₄alkyl;

B is selected from

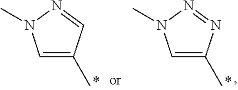

wherein the bond marked with * is attached to the carboxamide group;

C is a 6-membered aromatic ring system, which may contain 1, 2, 3 or 4 heteroatoms selected from N, O and S, wherein the ring system C is substituted once by R₆;

wherein R₆ is selected from halogen, C₁-C₄alkyl, C₁-C₄alkoxy, C₁-C₄halogenalkyl or —(C═NH)NH₂.

In certain embodiments, the invention relates to a compound of formula (I) in free form or in pharmaceutically acceptable salt form, in which:
(1) R₁ is hydrogen;
(2) R₁ is methyl;
(3) R₂ is hydrogen;
(4) R₂ is methyl;
(5) A is a 6-membered monocyclic aromatic ring system which may contain 1, 2, 3 or 4 heteroatoms selected from N, O and S, wherein A is neither unsubstituted phenyl nor unsubstituted pyridinyl.
(6) A is a 9-membered fused bicyclic aromatic ring system which contains 1, 2, 3, or 4 heteroatoms selected from N, O and S;
(7) A is a 10-membered fused bicyclic aromatic ring system which may contain 1, 2, 3, or 4 heteroatoms selected from N, O and S;
(8) A is phenyl substituted once or twice by R₃;
(9) A is naphthyl;
(10) A is quinolinyl;
(11) R₃ is halogen;
(12) R₃ is chloro;
(13) R₃ is fluoro;
(14) R₃ is C₁-C₄alkyl;
(15) R₃ is methyl;
(16) R₃ is ethyl;
(17) R₃ is C₁-C₄alkoxy;
(18) R₃ is methoxy;
(19) R₃ is ethoxy;
(20) R₃ is oxo;
(21) R₃ is cyano;
(22) R₃ is C₁-C₄halogenalkyl;
(23) R₃ is trifluoromethyl;
(24) R₃ is NH₂;
(25) R₃ is NH(CH₃);
(26) R₃ is N(CH₃)₂;
(27) R₃ is a 5-membered aromatic or non-aromatic ring system which contains 1, 2, 3, or 4 heteroatoms selected from N, O and S, which is attached to A via methylene or SO₂;
(28) R₃ is a 6-membered aromatic ring system which may contain 1, 2, 3, or 4 heteroatoms selected from N, O and S, which is optionally substituted by oxo, which is attached to A via methylene or SO₂;
(29) R₃ is pyridinyl;
(30) R₃ is oxazolidinyl;
(31) R₃ is pyrazolyl;
(32) R₃ is pyrrolyl;
(33) B is pyrazolyl;
(34) B is triazolyl;
(35) C is phenyl;
(36) C is pyridinyl, wherein C is neither 2-aminopyridinyl nor 6-aminopyridinyl;
(37) C is pyrrolopyrimidyl;
(38) C is indolyl;
(39) C is indazolyl;
(40) C is isoquinolinyl;
(41) C is naphthyl;
(42) C is benzothiophenyl;
(43) C is pyrazinyl;
(44) R₆ is chloro;
(45) R₆ is fluoro;
(46) R₆ is methyl;
(47) R₆ is ethyl;
(48) R₆ is methoxy;
(49) R₆ is ethoxy;
(50) R₆ is trifluoromethyl;
(51) R₆ is (C═NH)NH₂;

The skilled person would understand that the embodiments (1) to (51) may be used independently, collectively or in any combination or sub-combination to limit the scope of the invention as described hereinbefore in relation to compounds of formula (I) as appropriate.

Preferably, the compounds of the invention are not N-((2-chloro-6-methylpyridin-3-yl)methyl)-1-((2-methylquinolin-6-yl)methyl)-1H-1,2,3-triazole-4-carboxamide, N-((6-methoxy-2,4-dimethylpyridin-3-yl)methyl)-1-((2-methylquinolin-6-yl)methyl)-1H-1,2,3-triazole-4-carboxamide, N-(4-chlorobenzyl)-1-(4-methoxybenzyl)-1H-pyrazole-4-carboxamide, N-(4-chlorobenzyl)-1-(3-methoxybenzyl)-1H-pyrazole-4-carboxamide and N-(3-chlorobenzyl)-1-((1-methyl-1H-benzo[d][1,2,3]triazol-5-yl)methyl)-1H-pyrazole-4-carboxamide.

In one embodiment, the invention provides a compound in free form or in pharmaceutically acceptable form which is selected from N-((2,4-dimethylpyridin-3-yl)methyl)-1-((2-methylquinolin-6-yl)methyl)-1H-1,2,3-triazole-4-carboxamide;
N-((4-methoxy-2-methylpyridin-3-yl)methyl)-1-((2-methylquinolin-6-yl)methyl)-1H-1,2,3-triazole-4-carboxamide;
N-((6-hydroxy-2,4-dimethylpyridin-3-yl)methyl)-1-((2-methylquinolin-6-yl)methyl)-1H-1,2,3-triazole-4-carboxamide;
N-((2-hydroxy-3,5-dimethylpyridin-4-yl)methyl)-1-((2-methylquinolin-6-yl)methyl)-1H-1,2,3-triazole-4-carboxamide;
N-((5-methyl-1H-pyrrolo[2,3-b]pyridin-3-yl)methyl)-1-((2-methylquinolin-6-yl)methyl)-1H-1,2,3-triazole-4-carboxamide;
N-((4-methyl-2-(trifluoromethyl)pyridin-3-yl)methyl)-1-((2-methylquinolin-6-yl)methyl)-1H-1,2,3-triazole-4-carboxamide;
1-(4-((1H-pyrazol-1-yl)methyl)benzyl)-N-(3-chlorobenzyl)-1H-pyrazole-4-carboxamide;
1-(4-((1H-pyrazol-1-yl)methyl)benzyl)-N-(1-(3-chlorophenyl)ethyl)-1H-pyrazole-4-carboxamide;
N-((5-chloro-1H-indazol-3-yl)methyl)-1-(4-methoxybenzyl)-1H-pyrazole-4-carboxamide;
N-((5-chloro-1H-indol-3-yl)methyl)-1-((2-methylquinolin-6-yl)methyl)-1H-pyrazole-4-carboxamide;
N-((5-amino-3-methylpyrazin-2-yl)methyl)-1-((2-methylquinolin-6-yl)methyl)-1H-1,2,3-triazole-4-carboxamide;
N-(6-chloro-2-fluoro-3-methoxybenzyl)-1-((2-methylquinolin-6-yl)methyl)-1H-1,2,3-triazole-4-carboxamide;
N-((5-chloro-1H-indazol-3-yl)methyl)-1-(4-((2-oxopyridin-1 (2H)-yl)methyl)benzyl)-1H-pyrazole-4-carboxamide;
N-((5-chloro-1H-indol-3-yl)methyl)-1-(4-((2-oxopyridin-1 (2H)-yl)methyl)benzyl)-1H-pyrazole-4-carboxamide;
1-(4-((1H-pyrazol-1-yl)methyl)benzyl)-N-((5-chloro-1H-indazol-3-yl)methyl)-1H-pyrazole-4-carboxamide;
1-(4-((1H-pyrazol-1-yl)methyl)benzyl)-N-((5-chloro-1H-indol-3-yl)methyl)-1H-pyrazole-4-carboxamide;
N-((5-chloro-1H-indazol-3-yl)methyl)-1-(4-((2-oxooxazolidin-3-yl)methyl)benzyl)-1H-pyrazole-4-carboxamide;
N-((5-chloro-1H-indazol-3-yl)methyl)-1-((2-methylquinolin-6-yl)methyl)-1H-pyrazole-4-carboxamide;
1-(4-((1H-pyrrol-1-yl)sulfonyl)benzyl)-N-((5-chloro-1H-indazol-3-yl)methyl)-1H-pyrazole-4-carboxamide;
N-((5-chloro-1H-indol-3-yl)methyl)-1-(4-((2-oxooxazolidin-3-yl)methyl)benzyl)-1H-pyrazole-4-carboxamide;
1-(4-((1H-pyrrol-1-yl)sulfonyl)benzyl)-N-((5-chloro-1H-indol-3-yl)methyl)-1H-pyrazole-4-carboxamide;
N-((5-chloro-1H-indazol-3-yl)methyl)-1-(3,4-dimethoxybenzyl)-1H-pyrazole-4-carboxamide;
N-((5-chloro-1H-indazol-3-yl)methyl)-1-(3,5-dimethoxybenzyl)-1H-pyrazole-4-carboxamide;
N-((5-chloro-1H-indazol-3-yl)methyl)-1-(3-methoxybenzyl)-1H-pyrazole-4-carboxamide;
1-(4-((1H-pyrazol-1-yl)methyl)benzyl)-N-(3-chloro-2-fluorobenzyl)-1H-pyrazole-4-carboxamide; and
1-(4-((1H-pyrazol-1-yl)methyl)benzyl)-N-(1-(3-chloro-2-fluorophenyl)ethyl)-1H-pyrazole-4-carboxamide.

In one embodiment, the invention provides a compound which is
N-(4-carbamimidoylbenzyl)-1-(naphthalen-2-ylmethyl)-1H-pyrazole-4-carboxamide hydrochloride; or
1-(4-((1H-pyrazol-1-yl)methyl)benzyl)-N-(4-carbamimidoylbenzyl)-1H-pyrazole-4-carboxamide hydrochloride.

In one embodiment, the invention provides a compound in free form or in pharmaceutically acceptable salt form which is
(R)-1-(4-((1H-pyrazol-1-yl)methyl)benzyl)-N-(1-(3-chlorophenyl)ethyl)-1H-pyrazole-4-carboxamide or
(R)-1-(4-((1H-pyrazol-1-yl)methyl)benzyl)-N-(1-(3-chloro-2-fluorophenyl)ethyl)-1H-pyrazole-4-carboxamide.

As used herein, the term “an optical isomer” or “a stereoisomer” refers to any of the various stereo isomeric configurations which may exist for a given compound of the present invention and includes geometric isomers. It is understood that a substituent may be attached at a chiral center of a carbon atom. The term “chiral” refers to molecules which have the property of non-superimposability on their mirror image partner, while the term “achiral” refers to molecules which are superimposable on their mirror image partner. Therefore, the invention includes enantiomers, diastereomers or racemates of the compound. “Enantiomers” are a pair of stereoisomers that are non- superimposable mirror images of each other. A 1:1 mixture of a pair of enantiomers is a “racemic” mixture. The term is used to designate a racemic mixture where appropriate. “Diastereoisomers” are stereoisomers that have at least two asymmetric atoms, but which are not mirror-images of each other. The absolute stereochemistry is specified according to the Cahn-Ingold-Prelog R—S system. When a compound is a pure enantiomer the stereochemistry at each chiral carbon may be specified by either R or S. Resolved compounds whose absolute configuration is unknown can be designated (+) or (−) depending on the direction (dextro- or levorotatory) which they rotate plane polarized light at the wavelength of the sodium D line. Certain compounds described herein may contain one or more asymmetric centers or axes and may thus give rise to enantiomers, diastereomers, and other stereoisomeric forms that may be defined, in terms of absolute stereochemistry, as (R)- or (S)-.

Depending on the choice of the starting materials and procedures, the compounds can be present in the form of one of the possible isomers or as mixtures thereof, for example as pure optical isomers, or as isomer mixtures, such as racemates and diastereoisomer mixtures, depending on the number of asymmetric carbon atoms. The present invention is meant to include all such possible isomers, including racemic mixtures, diastereomeric mixtures and optically pure forms. Optically active (R)- and (S)-isomers may be prepared using chiral synthons or chiral reagents, or resolved using conventional techniques. If the compound contains a double bond, the substituent may be E or Z configuration. If the compound contains a disubstituted cycloalkyl, the cycloalkyl substituent may have a cis- or trans-configuration. All tautomeric forms are also intended to be included.

If present, any asymmetric atom (e.g., carbon or the like) of the compound(s) of the present invention can be present in racemic or enantiomerically enriched, for example the (R)-, (S)- or (R,S)-configuration. In certain embodiments, each asymmetric atom has at least 50% enantiomeric excess, at least 60% enantiomeric excess, at least 70% enantiomeric excess, at least 80% enantiomeric excess, at least 90% enantiomeric excess, at least 95% enantiomeric excess, or at least 99% enantiomeric excess in the (R)- or (S)-configuration. Substituents at atoms with unsaturated double bonds may, if possible, be present in cis-(Z)- or trans-(E)-form.

Accordingly, as used herein a compound of the present invention can be in the form of one of the possible isomers, rotamers, atropisomers, tautomers or mixtures thereof, for example, as substantially pure geometric (cis or trans) isomers, diastereomers, optical isomers (antipodes), racemates or mixtures thereof.

Any resulting mixtures of isomers can be separated on the basis of the physicochemical differences of the constituents, into the pure or substantially pure geometric or optical isomers, diastereomers, racemates, for example, by chromatography and/or fractional crystallization.

Any resulting racemates of final products or intermediates can be resolved into the optical antipodes by known methods, e.g., by separation of the diastereomeric salts thereof, obtained with an optically active acid or base, and liberating the optically active acidic or basic compound. In particular, a basic moiety may thus be employed to resolve the compounds of the present invention into their optical antipodes, e.g., by fractional crystallization of a salt formed with an optically active acid, e.g., tartaric acid, dibenzoyl tartaric acid, diacetyl tartaric acid, di-O,O′-p-toluoyl tartaric acid, mandelic acid, malic acid or camphor-10-sulfonic acid. Racemic products can also be resolved by chiral chromatography, e.g., high pressure liquid chromatography (HPLC) using a chiral adsorbent.

As used herein, the terms “salt” or “salts” refers to an acid addition or base addition salt of a compound of the invention. “Salts” include in particular “pharmaceutical acceptable salts”. The term “pharmaceutically acceptable salts” refers to salts that retain the biological effectiveness and properties of the compounds of this invention and, which typically are not biologically or otherwise undesirable. In many cases, the compounds of the present invention are capable of forming acid and/or base salts by virtue of the presence of amino and/or carboxyl groups or groups similar thereto.

Pharmaceutically acceptable acid addition salts can be formed with inorganic acids and organic acids, e.g., acetate, aspartate, benzoate, besylate, bromide/hydrobromide, bicarbonate/carbonate, bisulfate/sulfate, camphorsulfonate, chloride/hydrochloride, chlortheophyllonate, citrate, ethandisulfonate, fumarate, gluceptate, gluconate, glucuronate, glycolate, hippurate, hydroiodide/iodide, isethionate, lactate, lactobionate, laurylsulfate, malate, maleate, malonate, mandelate, mesylate, methylsulphate, naphthoate, napsylate, nicotinate, nitrate, octadecanoate, oleate, oxalate, palmitate, pamoate, phosphate/hydrogen phosphate/dihydrogen phosphate, polygalacturonate, propionate, stearate, succinate, sulfosalicylate, tartrate, tosylate and trifluoroacetate salts.

Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, oxalic acid, maleic acid, malonic acid, succinic acid, fumaric acid, tartaric acid, citric acid, benzoic acid, mandelic acid, methanesulfonic acid, ethanesulfonic acid, toluenesulfonic acid, sulfosalicylic acid, and the like. Pharmaceutically acceptable base addition salts can be formed with inorganic and organic bases.

Inorganic bases from which salts can be derived include, for example, ammonium salts and metals from columns I to XII of the periodic table. In certain embodiments, the salts are derived from sodium, potassium, ammonium, calcium, magnesium, iron, silver, zinc, and copper; particularly suitable salts include ammonium, potassium, sodium, calcium and magnesium salts.

Organic bases from which salts can be derived include, for example, primary, secondary, and tertiary amines, substituted amines including naturally occurring substituted amines, cyclic amines, basic ion exchange resins, and the like. Certain organic amines include isopropylamine, benzathine, cholinate, diethanolamine, diethylamine, lysine, meglumine, piperazine and tromethamine.

The pharmaceutically acceptable salts of the present invention can be synthesized from a basic or acidic moiety, by conventional chemical methods. Generally, such salts can be prepared by reacting free acid forms of these compounds with a stoichiometric amount of the appropriate base (such as Na, Ca, Mg, or K hydroxide, carbonate, bicarbonate or the like), or by reacting free base forms of these compounds with a stoichiometric amount of the appropriate acid. Such reactions are typically carried out in water or in an organic solvent, or in a mixture of the two. Generally, use of non-aqueous media like ether, ethyl acetate, ethanol, isopropanol, or acetonitrile is desirable, where practicable. Lists of additional suitable salts can be found, e.g., in “Remington's Pharmaceutical Sciences”, 20th ed., Mack Publishing Company, Easton, Pa., (1985); and in “Handbook of Pharmaceutical Salts Properties, Selection, and Use” by Stahl and Wermuth (Wiley-VCH, Weinheim, Germany, 2002).

Any formula given herein is also intended to represent unlabeled forms as well as isotopically labeled forms of the compounds. Isotopically labeled compounds have structures depicted by the formulae given herein except that one or more atoms are replaced by an atom having a selected atomic mass or mass number. Examples of isotopes that can be incorporated into compounds of the invention include isotopes of hydrogen, carbon, nitrogen, oxygen, phosphorous, fluorine, and chlorine, such as ²H, ³H, ¹¹C, ¹³C, ¹⁴C, ¹⁵N, ¹⁸F, ³¹P, ³²P, ³⁵S, ³⁶Cl, ¹²⁵I respectively. The invention includes various isotopically labeled compounds as defined herein, for example those into which radioactive isotopes, such as ³H and ¹⁴C, or those into which non-radioactive isotopes, such as ²H and ¹³C are present. Such isotopically labelled compounds are useful in metabolic studies (with ¹⁴C), reaction kinetic studies (with, for example ²H or ³H), detection or imaging techniques, such as positron emission tomography (PET) or single-photon emission computed tomography (SPECT) including drug or substrate tissue distribution assays, or in radioactive treatment of patients. In particular, an ¹⁸F or labeled compound may be particularly desirable for PET or SPECT studies. Isotopically-labeled compounds of formula (I) can generally be prepared by conventional techniques known to those skilled in the art or by processes analogous to those described in the accompanying Examples and Preparations using an appropriate isotopically-labeled reagents in place of the non-labeled reagent previously employed.

Further, substitution with heavier isotopes, particularly deuterium (i.e., ²H or D) may afford certain therapeutic advantages resulting from greater metabolic stability, for example increased in vivo half-life or reduced dosage requirements or an improvement in therapeutic index. It is understood that deuterium in this context is regarded as a substituent of a compound of the formula (I). The concentration of such a heavier isotope, specifically deuterium, may be defined by the isotopic enrichment factor. The term “isotopic enrichment factor” as used herein means the ratio between the isotopic abundance and the natural abundance of a specified isotope. If a substituent in a compound of this invention is denoted deuterium, such compound has an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation).

Pharmaceutically acceptable solvates in accordance with the invention include those wherein the solvent of crystallization may be isotopically substituted, e.g. D₂O, d₆-acetone, d₆-DMSO.

Compounds of the invention, i.e. compounds of formula (I) that contain groups capable of acting as donors and/or acceptors for hydrogen bonds may be capable of forming co-crystals with suitable co-crystal formers. These co-crystals may be prepared from compounds of formula (I) by known co-crystal forming procedures. Such procedures include grinding, heating, co-subliming, co-melting, or contacting in solution compounds of formula (I) with the co-crystal former under crystallization conditions and isolating co-crystals thereby formed. Suitable co-crystal formers include those described in WO 2004/078163. Hence the invention further provides co-crystals comprising a compound of formula (I).

As used herein, the term “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drug stabilizers, binders, excipients, disintegration agents, lubricants, sweetening agents, flavoring agents, dyes, and the like and combinations thereof, as would be known to those skilled in the art (see, for example, Remington's Pharmaceutical Sciences, 18th Ed. Mack Printing Company, 1990, pp. 1289-1329). Except insofar as any conventional carrier is incompatible with the active ingredient, its use in the therapeutic or pharmaceutical compositions is contemplated.

Furthermore, the compounds of the present invention, including their salts, can also be obtained in the form of their hydrates, or include other solvents used for their crystallization. The compounds of the present invention may inherently or by design form solvates with pharmaceutically acceptable solvents (including water); therefore, it is intended that the invention embrace both solvated and unsolvated forms. The term “solvate” refers to a molecular complex of a compound of the present invention (including pharmaceutically acceptable salts thereof) with one or more solvent molecules. Such solvent molecules are those commonly used in the pharmaceutical art, which are known to be innocuous to the recipient, e.g., water, ethanol, and the like. The term “hydrate” refers to the complex where the solvent molecule is water.

The compounds of the present invention, including salts, hydrates and solvates thereof, may inherently or by design form polymorphs.

Typically, the compounds of formula (I) can be prepared according to the Scheme provided infra.

In a further aspect, the invention also provides a process for the production of compounds of the formula (I). Compounds of the formula (I) are obtainable according to the following process as described in scheme 1:

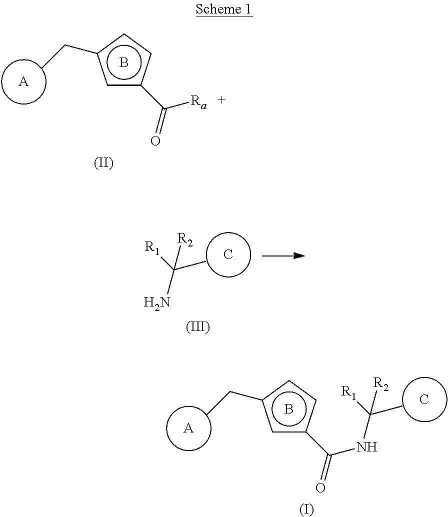

A compound of formula (I) wherein A, B, C, R₁ and R₂ are as defined herein may be obtained by reacting a compound of formula (II) wherein A and B are as defined herein in relation to compounds of formula (I) and Rₐ is a suitable group such as hydroxy with an amine of formula (III) wherein C, R₁ and R₂ are as defined herein in relation to compounds of formula (I) in the presence of a coupling agent such as e.g. O-(benzotriazol-1-yl)-N,N,N′,N′-tetramethyluronium hexafluorophosphate (HBTU) or an anhydride such as e.g. propylphosphonic anhydride in the presence of a base such as e.g. N,N-diisopropylethylamine (DIPEA), in a suitable solvent, e.g. dimethylformamide (DMF) or dichloromethane (DCM).

In another aspect, the present invention provides a pharmaceutical composition comprising a compound of the present invention, or a pharmaceutically acceptable salt thereof, and a pharmaceutically acceptable carrier. In particular, the present invention provides a pharmaceutical composition comprising a therapeutically effective amount of a compound of the invention in free form or in pharmaceutically acceptable salt form and one or more pharmaceutically acceptable carriers. The pharmaceutical composition can be formulated for particular routes of administration such as oral administration, parenteral administration, and rectal administration, etc. In addition, the pharmaceutical compositions of the present invention can be made up in a solid form (including without limitation capsules, tablets, pills, granules, powders or suppositories), or in a liquid form (including without limitation solutions, suspensions or emulsions). The pharmaceutical compositions can be subjected to conventional pharmaceutical operations such as sterilization and/or can contain conventional inert diluents, lubricating agents, or buffering agents, as well as adjuvants, such as preservatives, stabilizers, wetting agents, emulsifiers and buffers, etc.

Typically, the pharmaceutical compositions are tablets or gelatin capsules comprising the active ingredient together with

a) diluents, e.g., lactose, dextrose, sucrose, mannitol, sorbitol, cellulose and/or glycine;

b) lubricants, e.g., silica, talcum, stearic acid, its magnesium or calcium salt and/or polyethyleneglycol; for tablets also

c) binders, e.g., magnesium aluminum silicate, starch paste, gelatin, tragacanth, methylcellulose, sodium carboxymethylcellulose and/or polyvinylpyrrolidone; if desired

d) disintegrants, e.g., starches, agar, alginic acid or its sodium salt, or effervescent mixtures; and/or

e) absorbents, colorants, flavors and sweeteners.

Tablets may be either film coated or enteric coated according to methods known in the art.

Suitable compositions for oral administration include an effective amount of a compound of the invention in the form of tablets, lozenges, aqueous or oily suspensions, dispersible powders or granules, emulsion, hard or soft capsules, or syrups or elixirs. Compositions intended for oral use are prepared according to any method known in the art for the manufacture of pharmaceutical compositions and such compositions can contain one or more agents selected from the group consisting of sweetening agents, flavoring agents, coloring agents and preserving agents in order to provide pharmaceutically elegant and palatable preparations. Tablets may contain the active ingredient in admixture with nontoxic pharmaceutically acceptable excipients which are suitable for the manufacture of tablets. These excipients are, for example, inert diluents, such as calcium carbonate, sodium carbonate, lactose, calcium phosphate or sodium phosphate; granulating and disintegrating agents, for example, corn starch, or alginic acid; binding agents, for example, starch, gelatin or acacia; and lubricating agents, for example magnesium stearate, stearic acid or talc. The tablets are uncoated or coated by known techniques to delay disintegration and absorption in the gastrointestinal tract and thereby provide a sustained action over a longer period. For example, a time delay material such as glyceryl monostearate or glyceryl distearate can be employed. Formulations for oral use can be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil.

Certain injectable compositions are aqueous isotonic solutions or suspensions, and suppositories are advantageously prepared from fatty emulsions or suspensions. Said compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. Said compositions are prepared according to conventional mixing, granulating or coating methods, respectively, and contain about 0.1-75%, or contain about 1-50%, of the active ingredient.

Suitable compositions for transdermal application include an effective amount of a compound of the invention with a suitable carrier. Carriers suitable for transdermal delivery include absorbable pharmacologically acceptable solvents to assist passage through the skin of the host. For example, transdermal devices are in the form of a bandage comprising a backing member, a reservoir containing the compound optionally with carriers, optionally a rate controlling barrier to deliver the compound of the skin of the host at a controlled and predetermined rate over a prolonged period of time, and means to secure the device to the skin.

Suitable compositions for topical application, e.g., to the skin and eyes, include aqueous solutions, suspensions, ointments, creams, gels or sprayable formulations, e.g., for delivery by aerosol or the like. Such topical delivery systems will in particular be appropriate for dermal application, e.g., for the treatment of skin cancer, e.g., for prophylactic use in sun creams, lotions, sprays and the like. They are thus particularly suited for use in topical, including cosmetic, formulations well-known in the art. Such may contain solubilizers, stabilizers, tonicity enhancing agents, buffers and preservatives.

As used herein a topical application may also pertain to an inhalation or to an intranasal application. They may be conveniently delivered in the form of a dry powder (either alone, as a mixture, for example a dry blend with lactose, or a mixed component particle, for example with phospholipids) from a dry powder inhaler or an aerosol spray presentation from a pressurised container, pump, spray, atomizer or nebuliser, with or without the use of a suitable propellant.

The present invention further provides anhydrous pharmaceutical compositions and dosage forms comprising the compounds of the present invention as active ingredients, since water may facilitate the degradation of certain compounds.

Anhydrous pharmaceutical compositions and dosage forms of the invention can be prepared using anhydrous or low moisture containing ingredients and low moisture or low humidity conditions. An anhydrous pharmaceutical composition may be prepared and stored such that its anhydrous nature is maintained. Accordingly, anhydrous compositions are packaged using materials known to prevent exposure to water such that they can be included in suitable formulary kits. Examples of suitable packaging include, but are not limited to, hermetically sealed foils, plastics, unit dose containers (e.g., vials), blister packs, and strip packs.

The invention further provides pharmaceutical compositions and dosage forms that comprise one or more agents that reduce the rate by which the compound of the present invention as an active ingredient will decompose. Such agents, which are referred to herein as “stabilizers,” include, but are not limited to, antioxidants such as ascorbic acid, pH buffers, or salt buffers, etc.

The compounds of formula I in free form or in salt form, exhibit valuable pharmacological properties, e.g. plasma kallikrein modulating properties, e.g. as indicated in in vitro tests as provided in the next sections, and are therefore indicated for therapy or for use as research chemicals, e.g. as tool compounds.

Compounds of the invention may be useful in the treatment of indications, such as: hereditary angioedema (HAE), retinopathy or diabetic retinopathy, proliferative and non-proliferative retinopathy, diabetic macular edema (DME), clinically significant macular edema (CSME), cystoid macular edema (CME), CME following cataract extraction, CME induced by cryotherapy, CME induced by uveitis, CME following vascular occlusion (e.g. central retina vein occlusion, branch retinal vein occlusion, or hemiretinal vein occlusion), retinal edema, complications related to cataract surgery in diabetic retinopathy, hypertensive retinopathy, retinal trauma, dry and wet aged-related macular degeneration (AMD), ischemic reperfusion injuries, e.g. in all kind of contexts associated with tissue and/or organ transplantation, surgically-induced brain injury, focal cerebral ischemia, global cerebral ischemia, glioma-associated edema, spinal cord injury, pain, ischemia, focal brain ischemia, neurological and cognitive deficits, deep vein thrombosis, stroke, myocardial infarction, acquired angioedema drug-related (ACE-inhibitors), edema, high altitude cerebral edema, cytotoxic cerebral edema, osmotic cerebral edema, obstructive hydrocephalus, radiation induced edema, lymph edema, traumatic brain injury, hemorrhagic stroke (e.g., cerebral stroke or subarachnoid stroke), intracerebral hemorrhage, hemorrhagic transformation of ischemic stroke, cerebral trauma associate with injury or surgery, brain aneurysm, arterio-venous malformation, reduction of blood losses during surgical procedures (e.g. cardiothoracic surgery, such as cardiopulmonary bypass or coronary artery bypass grafting), blood coagulation disorders such as thrombosis, itch, disorders with an inflammation component (such as multiple sclerosis), epilepsy, encephalitis, Alzheimer's disease, excessive daytime sleepiness, essential hypertension, increased blood pressure associated with diabetes or hyperlipidemia, renal insufficiency, chronic kidney disease, heart failure, microalbuminuria, albuminuria, proteinuria, disorders associated with increased vascular permeability (e.g. increased retinal vascular permeability, increased leg, feet, ankle vascular permeability), cerebral hemorrhage, microalbuminuria, albuminuria and proteinuria, deep vein thrombosis, coagulation from post fibrinolytic treatments, angina, angioedema, sepsis, arthritis (e.g. rheumatoid arthritis, osteoarthritis, infection arthritis), lupus, gout, psoriasis, blood loss during cardiopulmonary bypass, inflammatory bowel, diabetes, diabetic complications, infectious diseases, astrocyte-activation related diseases (e.g. Alzheimer's disease or multiple sclerosis), Parkinson's disease, amyotrophic lateral sclerosis, Creutzfeld-Jacob disease, stroke, epilepsy and trauma (e.g. brain trauma), allergic edema e.g. airflow obstruction in chronic allergic sinusitis or perennial rhinitis; airflow obstruction in acute asthma; serositis associated with systemic lupus erythematosus (SLE) and other diseases.

Compounds of the invention may be especially useful in the treatment of an indication selected from: retinopathy and edema-associated diseases.

Thus, as a further embodiment, the invention provides the use of a compound of formula (I) in free form or in pharmaceutically acceptable salt form as a medicament.

As a further embodiment, the invention provides the use of a compound of formula (I) in free form or in pharmaceutically acceptable salt form in therapy.

In a further embodiment, the therapy is selected from a disease which is ameliorated by inhibition of plasmakallikrein. In another embodiment, the disease is selected from the afore-mentioned list, e.g. retinopathy and edema-associated diseases.

In another embodiment, the invention provides a method of treating a disease which is ameliorated by inhibition of plasmakallikrein comprising administration of a therapeutically acceptable amount of a compound of formula (I) in free form or in pharmaceutically acceptable salt form. In a further embodiment, the disease is selected from the afore-mentioned list, suitably retinopathy and edema-associated diseases.

In one embodiment, the invention provides a method of inhibiting plasmakallikrein in a subject, wherein the method comprises administering to the subject a therapeutically effective amount of a compound of formula I.

In a further embodiment, the invention provides a method of treating a disorder or a disease in a subject mediated by plasmakallikrein, wherein the method comprises administering to the subject a therapeutically effective amount of a compound of formula I. Preferably said disorder or said disease is selected from retinopathy and edema-associated diseases.

In yet a further embodiment, the invention provides the use of a compound of formula I, for the treatment of a disorder or disease in a subject mediated by plasmakallikrein.

In yet a further embodiment, the invention provides the use of a compound of formula I, for the treatment of a disorder or disease in a subject characterized by an abnormal activity of plasmakallikrein. Preferably said disorder or said disease is selected from retinopathy and edema-associated diseases.

The term “a therapeutically effective amount” of a compound of the invention refers to an amount of the compound of the invention that will elicit the biological or medical response of a subject, for example, reduction or inhibition of an enzyme or a protein activity, or ameliorate symptoms, alleviate conditions, slow or delay disease progression, or prevent a disease, etc. In one non-limiting embodiment, the term “a therapeutically effective amount” refers to the amount of the compound of the invention that, when administered to a subject, is effective to (1) at least partially alleviating, inhibiting, preventing and/or ameliorating a condition, or a disorder or a disease (i) mediated by plasmakallikrein, or (ii) associated with plasmakallikrein activity, or (iii) characterized by abnormal activity of plasmakallikrein; or (2) reducing or inhibiting the activity of plasmakallikrein; or (3) reducing or inhibiting the expression of plasmakallikrein. In another non-limiting embodiment, the term “a therapeutically effective amount” refers to the amount of the compound of the invention that, when administered to a cell, or a tissue, or a non-cellular biological material, or a medium, is effective to at least partially reducing or inhibiting the activity of plasmakallikrein; or at least partially reducing or inhibiting the expression of plasmakallikrein.

As used herein, the term “subject” refers to an animal. Preferably, the animal is a mammal. A subject also refers to for example, primates (e.g., humans), cows, sheep, goats, horses, dogs, cats, rabbits, rats, mice, fish, birds and the like. In a preferred embodiment, the subject is a human.

As used herein, the term “inhibition” or “inhibiting” refers to the reduction or suppression of a given condition, symptom, or disorder, or disease, or a significant decrease in the baseline activity of a biological activity or process.

As used herein, the term “treating” or “treatment” of any disease or disorder refers in one embodiment, to ameliorating the disease or disorder (i.e., slowing or arresting or reducing the development of the disease or at least one of the clinical symptoms thereof). In another embodiment “treating” or “treatment” refers to alleviating or ameliorating at least one physical parameter including those which may not be discernible by the patient. In yet another embodiment, “treating” or “treatment” refers to modulating the disease or disorder, either physically, (e.g., stabilization of a discernible symptom), physiologically, (e.g., stabilization of a physical parameter), or both. In yet another embodiment, “treating” or “treatment” refers to preventing or delaying the onset or development or progression of the disease or disorder.

As used herein, a subject is “in need of” a treatment if such subject would benefit biologically, medically or in quality of life from such treatment.

As used herein, the term “a,” “an,” “the” and similar terms used in the context of the present invention (especially in the context of the claims) are to be construed to cover both the singular and plural unless otherwise indicated herein or clearly contradicted by the context.

All methods described herein can be performed in any suitable order unless otherwise indicated herein or otherwise clearly contradicted by context. The use of any and all examples, or exemplary language (e.g. “such as”) provided herein is intended merely to better illuminate the invention and does not pose a limitation on the scope of the invention otherwise claimed.

The compound of the present invention may be administered either simultaneously with, or before or after, one or more other therapeutic agent. The compound of the present invention may be administered separately, by the same or different route of administration, or together in the same pharmaceutical composition as the other agents.

In one embodiment, the invention provides a combination comprising a therapeutically effective amount of a compound of the invention in free form or in pharmaceutically acceptable salt form and one or more therapeutically active agents.

In one embodiment, the invention provides a product comprising a compound of formula (I) and at least one other therapeutic agent as a combined preparation for simultaneous, separate or sequential use in therapy. In one embodiment, the therapy is the treatment of a disease or condition mediated by plasma kallikrein inhibition. Products provided as a combined preparation include a composition comprising the compound of formula (I) and the other therapeutic agent(s) together in the same pharmaceutical composition, or the compound of formula (I) and the other therapeutic agent(s) in separate form, e.g. in the form of a kit.

In one embodiment, the invention provides a pharmaceutical composition comprising a compound of formula (I) and another therapeutic agent(s). Optionally, the pharmaceutical composition may comprise a pharmaceutically acceptable carrier, as described above.

In one embodiment, the invention provides a kit comprising two or more separate pharmaceutical compositions, at least one of which contains a compound of formula (I). In one embodiment, the kit comprises means for separately retaining said compositions, such as a container, divided bottle, or divided foil packet. An example of such a kit is a blister pack, as typically used for the packaging of tablets, capsules and the like.

The kit of the invention may be used for administering different dosage forms, for example, oral and parenteral, for administering the separate compositions at different dosage intervals, or for titrating the separate compositions against one another. To assist compliance, the kit of the invention typically comprises directions for administration.

In the combination therapies of the invention, the compound of the invention and the other therapeutic agent may be manufactured and/or formulated by the same or different manufacturers. Moreover, the compound of the invention and the other therapeutic may be brought together into a combination therapy: (i) prior to release of the combination product to physicians (e.g. in the case of a kit comprising the compound of the invention and the other therapeutic agent); (ii) by the physician themselves (or under the guidance of the physician) shortly before administration; (iii) in the patient themselves, e.g. during sequential administration of the compound of the invention and the other therapeutic agent.

Accordingly, the invention provides the use of a compound of formula (I) for treating a disease or condition mediated by plasma kallikrein inhibition, wherein the medicament is prepared for administration with another therapeutic agent. The invention also provides the use of another therapeutic agent for treating a disease or condition mediated by plasma kallikrein inhibition, wherein the medicament is administered with a compound of formula (I).

The invention also provides a compound of formula (I) for use in a method of treating a disease or condition mediated by plasma kallikrein inhibition, wherein the compound of formula (I) is prepared for administration with another therapeutic agent.

The invention also provides another therapeutic agent for use in a method of treating a disease or condition mediated by plasma kallikrein inhibition, wherein the other therapeutic agent is prepared for administration with a compound of formula (I).

The invention also provides a compound of formula (I) for use in a method of treating a disease or condition mediated by plasma kallikrein inhibition, wherein the compound of formula (I) is administered with another therapeutic agent.

The invention also provides another therapeutic agent for use in a method of treating a disease or condition mediated by plasma kallikrein inhibition, wherein the other therapeutic agent is administered with a compound of formula (I).

The invention also provides the use of a compound of formula (I) for treating a disease or condition mediated by plasma kallikrein, wherein the patient has previously (e.g. within 24 hours) been treated with another therapeutic agent. The invention also provides the use of another therapeutic agent for treating a disease or condition mediated by plasma kallikrein, wherein the patient has previously (e.g. within 24 hours) been treated with a compound of formula (I).

The pharmaceutical composition or combination of the present invention can be in unit dosage of about 1-1000 mg of active ingredient(s) for a subject of about 50-70 kg, or about 1-500 mg or about 1-250 mg or about 1-150 mg or about 0.5-100 mg, or about 1-50 mg of active ingredients. The therapeutically effective dosage of a compound, the pharmaceutical composition, or the combinations thereof, is dependent on the species of the subject, the body weight, age and individual condition, the disorder or disease or the severity thereof being treated. A physician, clinician or veterinarian of ordinary skill can readily determine the effective amount of each of the active ingredients necessary to prevent, treat or inhibit the progress of the disorder or disease.

The above-cited dosage properties are demonstrable in vitro and in vivo tests using advantageously mammals, e.g., mice, rats, dogs, monkeys or isolated organs, tissues and preparations thereof. The compounds of the present invention can be applied in vitro in the form of solutions, e.g., aqueous solutions, and in vivo either enterally, parenterally, advantageously intravenously, e.g., as a suspension or in aqueous solution. The dosage in vitro may range between about 10⁻³ molar and 10⁻⁹ molar concentrations. A therapeutically effective amount in vivo may range depending on the route of administration, between about 0.1-500 mg/kg, or between about 1-100 mg/kg.

The activity of a compound according to the present invention can be assessed by the following in vitro method described in example 29.

The following examples are intended to illustrate the invention and are not to be construed as being limitations thereon. Temperatures are given in degrees Celsius. If not mentioned otherwise, all evaporations are performed under reduced pressure, typically between about 15 mm Hg and 100 mm Hg (=20-133 mbar). The structure of final products, intermediates and starting materials is confirmed by standard analytical methods, e.g., microanalysis and spectroscopic characteristics, e.g., MS, IR, NMR. Abbreviations used are those conventional in the art.

All starting materials, building blocks, reagents, acids, bases, dehydrating agents, solvents, and catalysts utilized to synthesis the compounds of the present invention are either commercially available or can be produced by organic synthesis methods known to one of ordinary skill in the art (Houben-Weyl 4th Ed. 1952, Methods of Organic Synthesis, Thieme, Volume 21). Further, the compounds of the present invention can be produced by organic synthesis methods known to one of ordinary skill in the art as shown in the following examples.

### EXAMPLES

Abbreviations:
ACN Acetonitrile
AcOH acetic acid
BEMP 2-tert-butylimino-2-diethylamino-1,3-dimethylperhydro-1,3,2-diazaphosphorine
br broad signal (NMR)
Cs₂CO₃ Cesium carbonate
DBU 1,8-diazabicyclo[5.4.0]undec-7-ene
DCM dichloromethane
DIBAL-H diisobutylaluminum hydride
DIPEA N,N-diisopropylethylamine
DMA N,N-dimethylacetamide
4-DMAP 4-dimethylaminopyridine
DMF dimethylformamide
DMSO dimethylsulfoxide
DPPA diphenyl phosphoryl azide
EDC.HCl 1-(3-dimethylaminopropyl)-3-ethylcarbodiimide.hydrochloride
EtOAc ethyl acetate
EtOH ethanol
h hour(s)
HBTU 0-(benzotriazol-1-yl)-N,N,N′,N′-tetramethyluronium hexafluorophosphate
HATU 0-(7-azabenzotriazol-1-yl)-N,N,N′,N′-tetramethyluronium hexafluorophosphate
HOAt 1-hydroxy-7-azabenzotriazole
HOBt 1-hydroxybenzotriazole
HPLC high pressure liquid chromatography
HV high vacuum
LHMDS lithium bis(trimethylsilyl)amide
LiOH Lithium hydroxide min minute(s)
MS mass spectrometry
MTBE tert-butyl methyl ether
NMR nuclear magnetic resonance spectroscopy
PTFA polytetrafluoroethylene
quant. Quantitative
R_{f} retention value (chromatography)
rt room temperature
Rtₓ retention time using method X (specified in experimental part)
TBME tert-butyl methyl ether
TEA triethylamine
TFA trifluoroacetic acid
THF tetrahydrofurane
TLC Thin Layer Chromatography

UPLC ultra performance liquid chromatography

### Experimental

TLC conditions: R_{f} values for TLC are measured on 5×10 cm TLC plates, silica gel F₂₅₄, Merck, Darmstadt, Germany.

1H NMR spectra were recorded using a Bruker avance 400 or a Bruker Avance DPX400 Spectrometer. HPLC was performed using an Agilent 1100 or 1200 series instrument. Mass spectra and LC/MS were determined using an Agilent 1100 series instrument, a UPLC-MS Waters Alliance 2690 instrument, or a UPLC-MS Waters Acquity SQD system.

Method A: HPLC Instrument: Agilent 1100 series; Column: Waters SunFire C18 2.5 μm 3*30 mm, Eluent A: water+0.1% TFA; B: ACN+0.1% TFA. Gradient 10 to 98% B in 2.5 min, Flow: 1.4 ml/min.

Method B: HPLC Instrument: Agilent 1200 series; Column: ECLIPSE XDB-C18 1.8 μm 2.1*30 mm, Eluent A: water+0.1% TFA; B: ACN+0.1% TFA. Gradient 5 to 100% B in 3 min, 100% B during 0.75 min, Flow: 0.6 ml/min.

Method C: UPLC-MS Instrument: Waters UPLC Acquity; column: Acquity HSS T3 1.8 μm 2.1* 50 mm at 50° C., Eluent A: water+0.05% HCOOH+3.75 mM ammonium acetate, B: ACN+0.04% HCOOH, Gradient: 2 to 98% B in 1.4 min, Flow: 1.2 mL/min.

Method D: LC-MS Instrument: Agilent 1100 series; column: Waters Sunfire C18 2.5 μm 3*30 mm, Eluent A: water+0.1% HCOOH; B: ACN+0.1% HCOOH, Gradient: 10 to 98% B in 2.5 min.

Method E: UPLC-MS Instrument: Waters UPLC Acquity; column: Acquity HSS T3 1.8 μm 2.1×50 mm at 50° C., Eluent A: water+0.05% HCOOH+3.75 mM ammonium acetate, B: ACN+0.04% HCOOH, Gradient: 10 to 95% B in 1.5 min, Flow: 1.2 mL/min.

Method F: HPLC Instrument: Agilent 1200 series; Column: ECLIPSE XDB-C18 1.8 μm 4.6*50 mm, Eluent A: water+0.1% TFA; B ACN+0.1% TFA. Gradient 5 to 100% B in 6 min, 100% B during 1.5 min, Flow: 1 ml/min.

Method G: HPLC Instrument: Agilent 1100 series; Column: Waters symmetry C18 3.5 μm 2.1*50 mm, Eluent A: water+0.1% TFA; B: ACN+0.1% TFA, Gradient 5 to 95% B in 3.5 min, 95% B during 1.5 min, Flow: 0.6 ml/min

Method H: UPLC-MS Instrument: Waters UPLC Acquity; column: Acquity HSS T3 1.8 μm 2.1×50 mm at 50° C., Eluent A: water+0.05% HCOOH+3.75 mM ammonium acetate, B: ACN+0.04% HCOOH, Gradient: 2 to 98% B in 9.4 min, Flow: 1.2 mL/min.

Method I: HPLC Instrument: Agilent 1100 series; Column: Ascentis Express C18 2.7 μm 2.1*30 mm at 50° C., Eluent A: water+0.05% TFA; B: ACN+0.04% TFA, Gradient 1 to 95% B in 2.2 min, 95 to 99% B in 0.7 min, Flow: 1.2 ml/min.

### Example 1

### N-((2,4-dimethylpyridin-3-yl)methyl)-1-((2-methylquinolin-6-yl)methyl)-1H-1,2,3-triazole-4-carboxamide

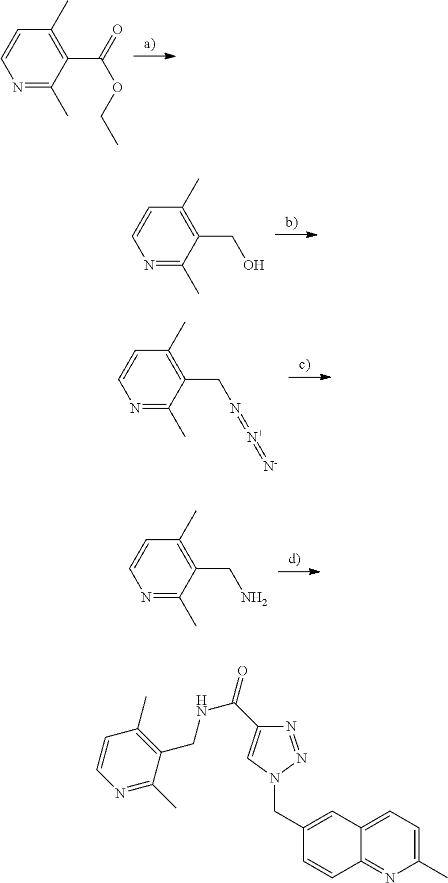

### a) (2,4-dimethylpyridin-3-yl)methanol

To a solution of ethyl 2,4-dimethylnicotinate (538 mg, 3 mmol) in DCM (5 ml) was added dropwise, at −70° C., DIBAL-H 1M in DCM (8 ml, 8 mmol). The reaction mixture was stirred at this temperature during 2 h. The reaction mixture was quenched by a slow addition of EtOAc (1 ml) and H₂O (1 ml), then concentrated. The crude residue was purified by flash chromatography on silica gel (gradient EtOAc then DCM/MeOH 95/5 to 9/1). TLC, Rf (DCM/MeOH 9/1)=0.22; [M+H]⁺=138.0.

### b) 3-(azidomethyl)-2,4-dimethylpyridine

To a solution of (2,4-dimethylpyridin-3-yl)methanol (255 mg, 1.859 mmol) in DMF (6 ml) was added, at 0° C., Diphenylphosphorylazide (1.023 g, 3.72 mmol) followed by DBU (0.84 ml, 5.58 mmol). The reaction mixture was stirred at rt overnight. The crude was poured into sat. aq. NaHCO₃ and extracted with EtOAc. The organic layer was dried (Na₂SO₄), filtered and concentrated. The crude residue was purified by flash chromatography on silica gel (EtOAc). [M+H]⁺=163.0. HPLC Rt_{F}=1.97 min.

### c) (2,4-dimethylpyridin-3-yl)methanamine

To a solution of 3-(azidomethyl)-2,4-dimethylpyridine (360 mg, 1.88 mmol) in MeOH (15 mL) was added Pd/C 10% (50 mg). The reaction was placed under hydrogen atmosphere and was stirred for 3 h. The catalyst was removed by filtration through a pad of Celite and washed with MeOH. Solvents were concentrated to give the title compound. [M+H]⁺=137.0.

### d) N-((2,4-dimethylpyridin-3-yl)methyl)-1-((2-methylquinolin-6-yl)methyl)-1H-1,2,3-triazole-4-carboxamide

To a solution of (2,4-dimethylpyridin-3-yl)methanamine (45 mg, 0.29 mmol) in DMF (2 ml) was added 1-((2-methylquinolin-6-yl)methyl)-1H-1,2,3-triazole-4-carboxylic acid (101 mg, 0.29 mmol), HATU (166 mg, 0.436 mmol) and DIPEA (0.2 ml, 1.16 mmol). The reaction mixture was stirred at rt overnight, purified by preparative HPLC (Macherey-Nagel Nucleosil 100-10 C18, flow: 50 mL/min, eluent: 5% to 99% ACN in H₂O in 20 min, ACN and H₂O containing 0.1% TFA). Pure HPLC fractions were neutralized with aq. sat. NaHCO₃, extracted with EtOAc. The organic layer was dried (Na₂SO₄), filtered and concentrated to give the desired material. [M+H]⁺=387.2, HPLC Rt_{B}=2.12 min, ¹H NMR (400 MHz, DMSO-d₆) δ ppm 2.36 (s, 3H) 2.53 (s, 3H) 2.64 (s, 3H) 4.48 (d, 2H) 5.82 (s, 2H) 7.03 (d, 1H) 7.43 (d, 1H) 7.65 (dd, 1H) 7.80-7.94 (m, 2H) 8.13-8.28 (m, 2H) 8.69 (s, 2H).

### Example 2

### N-((4-methoxy-2-methylpyridin-3-yl)methyl)-1-((2-methylquinolin-6-yl)methyl)-1H-1,2,3-triazole-4-carboxamide

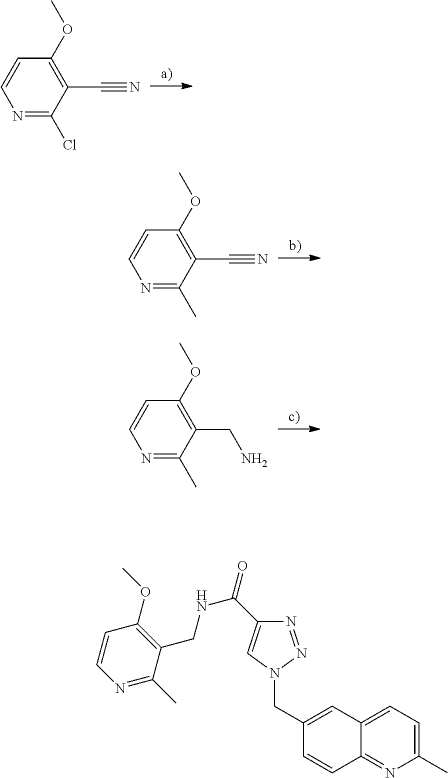

### a) 4-methoxy-2-methylnicotinonitrile

The title compound was prepared in a similar manner as described in Tetrahedron, 2006, 62, 6222-6227: To a solution of 2-chloro-4-methoxynicotinonitrile (350 mg, 2.076 mmol) in dioxane (8 ml) was added trimethylaluminium 2M in heptane (2.076 ml, 4.15 mmol) and Tetrakis(triphenylphosphine)palladium (36 mg, 0.031 mmol). The reaction mixture was heated to 70° C. for 1 h. Additional trimethylaluminium 2M in heptane (3 ml, 6 mmol) and Tetrakis(triphenylphosphine)palladium (36 mg, 0.031 mmol) were added and the reaction mixture was heated to 80° C. overnight. Another portion of trimethylaluminium 2M in heptane (3 ml, 6 mmol) and Tetrakis(triphenylphosphine)palladium (36 mg, 0.031 mmol) were added and the reaction mixture was heated to 90° C. during 8 h. After cooling to rt, the reaction mixture was poured dropwise into an aqueous Rochelle Salt 30% solution and stirred at rt 1 h. The mixture was extracted with EtOAc. The combined organic layers were dried (MgSO₄), filtered and concentrated. Purification by flash chromatography on silica gel (EtOAc) afforded desired compound. UPLC Rt_{C}=1.18 min, [M+H]⁺=149.1, HPLC Rt_{B}=0.36 min.

### b) (4-methoxy-2-methylpyridin-3-yl)methanamine hydrochloride

To a solution of 4-methoxy-2-methylnicotinonitrile (145 mg, 0.94 mmol) in MeOH (7 ml), EtOH (7 ml) and HCl 1N (5 ml) was added Pd/C 10% (50 mg). The reaction was placed under hydrogen atmosphere and was stirred overnight at rt. The catalyst was removed by filtration through a pad of Celite and washed with MeOH. Solvents were concentrated to give the title compound. [M+H]⁺=153.0.

### c) N-((4-methoxy-2-methylpyridin-3-yl)methyl)-1-((2-methylquinolin-6-yl)methyl)-1H-1,2,3-triazole-4-carboxamide

To a solution of (4-methoxy-2-methylpyridin-3-yl)methanamine hydrochloride (55 mg, 0.274 mmol) in DCM (5 ml) was added 1-((2-methylquinolin-6-yl)methyl)-1H-1,2,3-triazole-4-carboxylic acid (95 mg, 0.274 mmol), HATU (156 mg, 0.411 mmol) and DIPEA (0.19 ml, 1.09 mmol). The reaction mixture was stirred at rt overnight. Volatiles were evaporated. The crude residue was purified by preparative HPLC (Macherey-Nagel Nucleosil 100-10 C18, flow: 50 mL/min, eluent: 5% to 99% ACN in H₂O in 20 min, ACN and H₂O containing 0.1% TFA). Pure HPLC fractions were neutralized with aq. sat. NaHCO₃, extracted with EtOAc, dried (Na₂SO₄), filtered and concentrated to give the desired material. [M+H]⁺=403.2, HPLC Rt_{B}=2.18 min. ¹H NMR (400 MHz, DMSO-d₆) δ ppm 2.64 (s, 3H) 3.83 (s, 3H) 4.47 (d, 2H) 5.81 (s, 2H) 6.90 (d, 1H) 7.42 (d, 1H) 7.64 (dd, 1H) 7.84 (s, 1H) 7.90 (d, 1H) 8.14-8.32 (m, 3H) 8.68 (s, 1H).

### Example 3

### N-((6-hydroxy-2,4-dimethylpyridin-3-yl)methyl)-1-((2-methylquinolin-6-yl)methyl)-1H-1,2,3-triazole-4-carboxamide

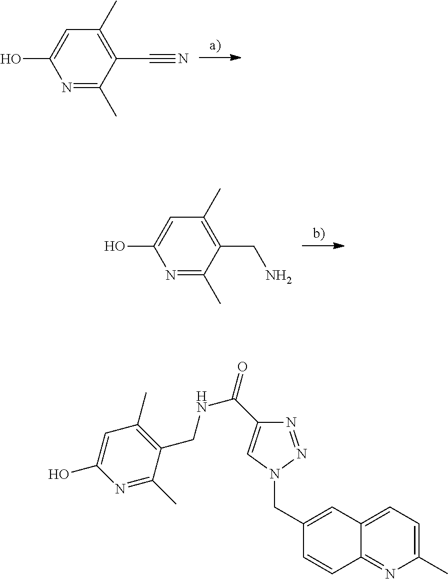 [SMILES: Cc1cc(O)nc(C)c1C#N ; Cc1cc(O)nc(C)c1CN ; Cc1ccc2cc(Cn3cc(C(=O)NCc4c(C)cc(O)nc4C)nn3)ccc2n1]

### a) 5-(aminomethyl)-4,6-dimethylpyridin-2-ol hydrochloride

To a solution of 6-hydroxy-2,4-dimethylnicotinonitrile (500 mg, 3.37 mmol) in MeOH (15 ml) and HCl 1N (10 ml) was added Pd/C 10% (50 mg). The reaction was placed under hydrogen atmosphere and was stirred overnight at rt. The catalyst was removed by filtration through a pad of Celite and washed with MeOH. Solvents were concentrated to give the title compound. [M+H]⁺=153.0.

### b) N-((6-hydroxy-2,4-dimethylqyridin-3-yl)methyl)-1-((2-methylquinolin-6-yl)methyl)-1H-1,2,3-triazole-4-carboxamide

To a suspension of 5-(aminomethyl)-4,6-dimethylpyridin-2-ol hydrochloride (81 mg, 0.382 mmol), 1-((2-methylquinolin-6-yl)methyl)-1H-1,2,3-triazole-4-carboxylic acid (90 mg, 0.258 mmol) and DIPEA (0.226 ml, 1.29 mmol) in DCM (5 ml) was added Propylphosphonic anhydride 50% in EtOAc (0.231 ml, 0.387 mmol). The reaction mixture was stirred at rt overnight, quenched with aq. sat. NaHCO₃ (25 ml) and EtOAc (25 ml). The suspension was filtered, the solid was washed with H₂O and EtOAc and dried on HV to obtain the title compound. UPLC Rt_{E}=0.44 min, [M+H]⁺=403.2; ¹H NMR (400 MHz, DMSO-d₆) δ ppm 2.14 (s, 3H) 2.26 (s, 3H) 2.64 (s, 3H) 4.21 (br. s., 2H) 5.82 (s, 2H) 5.97 (s, 1H) 7.43 (d, 1H) 7.65 (dd, 1H) 7.85 (s, 1H) 7.91 (d, 1H) 8.24 (d, 1H) 8.52 (br. s., 1H) 8.68 (s, 1H).

### Example 4

### N-((2-hydroxy-3,5-dimethylpyridin-4-yl)methyl)-1-((2-methylquinolin-6-yl)methyl)-1H-1,2,3-triazole-4-carboxamide

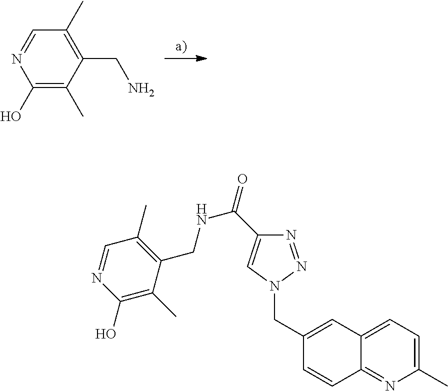 [SMILES: Cc1cnc(O)c(C)c1CN ; Cc1ccc2cc(Cn3cc(C(=O)NCc4c(C)cnc(O)c4C)nn3)ccc2n1]

### a) N-((2-hydroxy-3,5-dimethylqyridin-4-yl)methyl)-1-((2-methylquinolin-6-yl)methyl)-1H-1,2,3-triazole-4-carboxamide

To a suspension of 1-((2-methylquinolin-6-yl)methyl)-1H-1,2,3-triazole-4-carboxylic acid (29 mg, 0.068 mmol, 63%), EDC.HCl (16.86 mg, 0.088 mmol) and HOAt (11.97 mg, 0.088 mmol) in DMF (0.75 ml) was added N-methylmorpholine (0.022 ml, 0.203 mmol) and the reaction mixture was stirred at rt during 1 h. Then 4-(aminomethyl)-3,5-dimethylpyridin-2-ol (purchased from PepTech Corp., Burlington, Mass., USA) (11.44 mg, 0.068 mmol) was added and the mixture was stirred 3 h at rt. Volatiles were evaporated. The crude residue was purified by preparative HPLC (X-Bridge C18 ODB 30×100 mm, 5 μm, flow: 45 mL/min, eluent: 20% to 99% ACN in H2O in 12 min, ACN and H₂O containing 7.3 mM NH₃). UPLC Rt_{C}=0.56 min, [M+H]⁺=403.4, HPLC Rt_{G}=2.57 min, ¹H NMR (400 MHz, DMSO-d₆) δ ppm 1.95-2.16 (m, 6H) 2.64 (s, 3H) 4.31 (d, 2H) 5.82 (s, 2H) 6.97 (s, 1H) 7.43 (d, 1H) 7.65 (d, 1H) 7.77-7.98 (m, 2H) 8.24 (d, 1H) 8.61 (br. s., 1H) 8.69 (s, 1H) 11.25 (br. s., 1H).

### Example 5

### N-((5-methyl-1H-pyrrolo[2,3-b]pyridin-3-yl)methyl)-1-((2-methylquinolin-6-yl)methyl)-1H-1,2,3-triazole-4-carboxamide

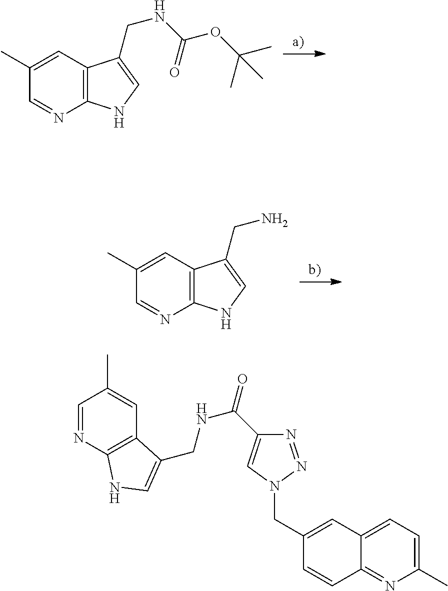

### a) (5-methyl-1H-pyrrolo[2,3-b]pyridin-3-yl)methanamine hydrochloride

tert-butyl (5-methyl-1H-pyrrolo[2,3-b]pyridin-3-yl)methylcarbamate (100 mg, 0.383 mol) was solved in HCl 4 M in dioxane (0.957 ml, 3.83 mmol). The reaction mixture was stirred at rt during 30 min. Volatiles were evaporated to give the title compound. This was further carried to the next step without further purification.

### b) N-((5-methyl-1H-pyrrolo[2,3-b]pyridin-3-yl)methyl)-1-((2-methylquinolin-6-yl)methyl)-1H-1,2,3-triazole-4-carboxamide

To a solution of (5-methyl-1H-pyrrolo[2,3-b]pyridin-3-yl)methanamine hydrochloride (76 mg, 0.384 mmol) and 1-((2-methylquinolin-6-yl)methyl)-1H-1,2,3-triazole-4-carboxylic acid (103 mg, 0.384 mmol) in DCM (2 ml) was added Propylphosphonic anhydride 50% in EtOAc (0.34 ml, 0.577 mmol) followed by DIPEA (0.336 ml, 1.922 mmol). The reaction mixture was stirred at rt during 30 min. The crude was diluted in DCM and washed with water and brine. The organic layer was dried (MgSO₄), filtered and concentrated. The residue was triturated with MeOH/ACN 1/1 (0.5 ml). The precipitate was filtered, washed with ACN and dried under HV. UPLC Rt_{H}=1.85 min, [M+H]⁺=412.3; HPLC Rt_{G}=2.05 min, ¹H NMR (400 MHz, DMSO-d₆) δ ppm 2.35 (s, 3H) 2.65 (s, 3H) 4.54 (d, 2H) 5.83 (s, 2H) 7.32 (d, 1H) 7.44 (d, 1H) 7.66 (dd, 1H) 7.84-7.93 (m, 3H) 8.04 (d, 1H) 8.25 (d, 1H) 8.72 (s, 1H) 8.85 (t, 1H) 11.29 (s, 1H).

### Example 6

### N-((4-methyl-2-(trifluoromethyl)pyridin-3-yl)methyl)-1-((2-methylquinolin-6-yl)methyl)-1H-1,2,3-triazole-4-carboxamide

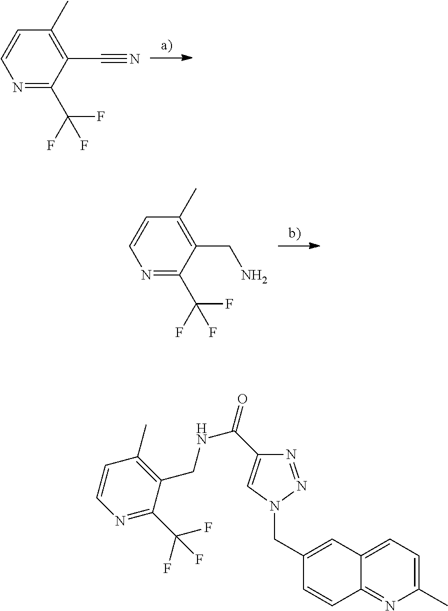 [SMILES: Cc1ccnc(C(F)(F)F)c1C#N ; Cc1ccnc(C(F)(F)F)c1CN ; Cc1ccc2cc(Cn3cc(C(=O)NCc4c(C)ccnc4C(F)(F)F)nn3)ccc2n1]

### a) (2-methyl-4-(trifluoromethyl)pyridin-3-yl)methanamine

To a solution of 2-methyl-4-(trifluoromethyl)nicotinonitrile (100 mg, 0.537 mmol) in 5% NH₃ in MeOH (15 ml) was added Raney/Nickel (40 mg). The reaction was placed under hydrogen atmosphere (3 bar) and was stirred at rt during 37 h. The catalyst was removed by filtration through a pad of Celite and washed with MeOH. Solvents were concentrated to give the title compound. UPLC Rt_{C}=0.74 min, [M+H]⁺=191.1.

### b) N-((4-methyl-2-(trifluoromethyl)pyridin-3-yl)methyl)-1-((2-methylquinolin-6-yl)methyl)-1H-1,2,3-triazole-4-carboxamide

To a suspension of (2-methyl-4-(trifluoromethyl)pyridin-3-yl)methanamine (110 mg, 0.463 mmol), 1-((2-methylquinolin-6-yl)methyl)-1H-1,2,3-triazole-4-carboxylic acid (124 mg, 0.463 mmol) and DIPEA (0.404 ml, 2.314 mmol) in DCM (3 ml) was added Propylphosphonic anhydride 50% in EtOAc (0.204 ml, 0.694 mmol). The reaction mixture was stirred at rt during 30 min, quenched with aq. sat. Na₂CO₃ and extracted with EtOAc (3×15 ml). The combined organic layers were dried (Na₂SO₄), filtered and concentrated. The residue was triturated with cyclohexane/AcOEt 2/1. The precipitate was filtered and dried on HV. UPLC Rt_{C}=0.77 min, [M+H]⁺=441.3, HPLC Rt_{G}=0.91 min, ¹H NMR (400 MHz, DMSO-d₆) δ ppm 2.56-2.70 (m, 6H) 4.61 (d, 2H) 5.84 (s, 2H) 7.44 (d, 1H) 7.57 (d, 1H) 7.67 (dd, 1H) 7.85-7.97 (m, 2H) 8.25 (d, 1H) 8.63-8.76 (m, 3H).

### Example 7

### 1-(4-((1H-pyrazol-1-yl)methyl)benzyl)-N-(3-chlorobenzyl)-1H-pyrazole-4-carboxamide

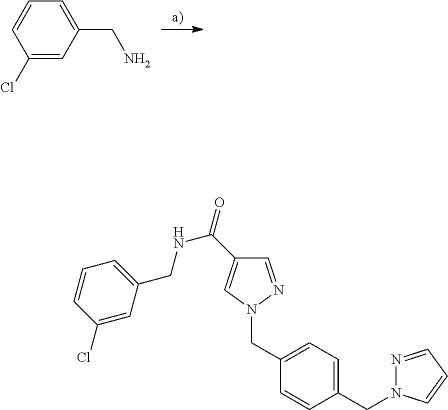 [SMILES: NCc1cccc(Cl)c1 ; O=C(NCc1cccc(Cl)c1)c1cnn(Cc2ccc(Cn3cccn3)cc2)c1]

### a) 1-(4-((1H-pyrazol-1-yl)methyl)benzyl)-N-(3-chlorobenzyl)-1H-pyrazole-4-carboxamide

1-(4-((1H-pyrazol-1-yl)methyl)benzyl)-1H-pyrazole-4-carboxylic acid (60 mg, 0.213 mmol) and (3-chlorophenyl)methanamine (31.6 mg, 0.223 mmol) were solved in DMF (1.5 ml) then DIPEA (0.148 ml, 0.85 mmol) and HATU (113 mg, 0.298 mmol) were added and the reaction mixture was stirred at rt overnight. The crude was purified by preparative HPLC (Waters Sunfire C18-OBD, 5 μm, 30×100 mm, flow: 40 mL/min, eluent: 5% to 80% ACN in H₂O in 20 min, ACN and H₂O containing 0.1% TFA). Pure HPLC fractions were neutralized with aq. sat. NaHCO₃. Volatiles were evaporated, the precipitate was filtered, washed with water and dried on HV to give the desired material. LCMS Rt_{D}=1.77 min, [M+H]⁺=406.0, [M+Na]+=427.9, HPLC Rt_{B}=3.43 min, ¹H NMR (500 MHz, DMSO-d₆) δ ppm 4.39 (d, 2H) 5.31 (d, 4H) 6.25 (t, 1H) 7.17-7.20 (m, 2H) 7.20-7.26 (m, 3H) 7.27-7.40 (m, 3H) 7.44 (d, 1H) 7.79 (d, 1H) 7.89 (s, 1H) 8.25 (s, 1H) 8.64 (t, 1H).

### Example 8

### (R)-1-(4-((1H-pyrazol-1-yl)methyl)benzyl)-N-(1-(3-chlorophenyl)ethyl)-1H-pyrazole-4-carboxamide

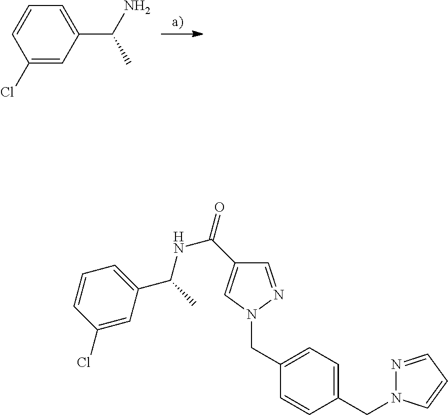

### a) (R)-1-(4-((1H-pyrazol-1-yl)methyl)benzyl)-N-(1-(3-chlorophenyl)ethyl)-1H-pyrazole-4-carboxamide

1-(4-((1H-pyrazol-1-yl)methyl)benzyl)-1H-pyrazole-4-carboxylic acid (50 mg, 0.177 mmol) and (R)-1-(3-chlorophenyl)ethanamine (27.8 mg, 0.177 mmol) were solved in DCM (2 ml) then DIPEA (0.046 ml, 0.266 mmol) and HATU (101 mg, 0.266 mmol) were added and the reaction mixture was stirred at rt during 5 h. The reaction mixture was poured into water and extracted with DCM (2×15 ml). The combined organic layers were dried (Na₂SO₄), filtered and concentrated. The residue was purified by preparative HPLC (Waters Sunfire C18-OBD, 5 μm, 30×100 mm, flow: 40 mL/min, eluent: 5% to 80% ACN in H₂O in 20 min, ACN and H₂O containing 0.1% TFA). Pure HPLC fractions were neutralized with aq. sat. NaHCO₃ and extracted with DCM, dried (Na₂SO₄), filtered and concentrated to give the desired material. UPLC Rt_{E}=0.95 min, [M+H]⁺=464.5, HPLC Rt_{A}=2.013 min, ¹H NMR (400 MHz, DMSO-d₆) δ ppm 1.40 (d, 3H) 5.07 (quin, 1H) 5.30 (s, 4H) 6.25 (t, 1H) 7.03-7.54 (m, 9H) 7.80 (d, 1H) 7.90 (s, 1H) 8.25 (s, 1H) 8.41 (d, 1H)

### Example 9

### N-(4-carbamimidoylbenzyl)-1-(naphthalen-2-ylmethyl)-1H-pyrazole-4-carboxamide hydrochloride

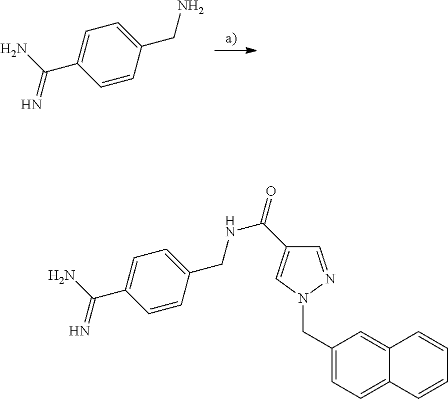 [SMILES: N=C(N)c1ccc(CN)cc1 ; N=C(N)c1ccc(CNC(=O)c2cnn(Cc3ccc4ccccc4c3)c2)cc1]

### a) N-(4-carbamimidoylbenzyl)-1-(naphthalen-2-ylmethyl)-1H-pyrazole-4-carboxamide hydrochloride

To a suspension of 1-(naphthalen-2-ylmethyl)-1H-pyrazole-4-carboxylic acid (1.0 g, 3.96 mmol), 4-(aminomethyl)benzimidamide dihydrochloride (1.10 g, 4.96 mmol) and DIPEA (4.15 ml, 23.78 mmol) in DMF (26.4 ml) was added Propylphosphonic anhydride 50% in EtOAc (2.92 ml, 4.96 mmol). The reaction mixture was stirred at rt for 6 h, purified by reverse phase flash chromatography (Silicycle C18, 40-63 μm, 190 g, 30×230 mm, flow: 80 mL/min, eluent: 3% to 98% MeOH in H₂O, MeOH and H₂O containing 0.1% TFA). Pure fractions were concentrated, made alkaline with 1N NaOH and extracted with DCM/MeOH 10/1. The combined organic layers were dried, filtered and concentrated. The residue was solved in MeOH (20 ml), filtered and the filtrate subjected on SCX-Isolute® SPE-column, washed with MeOH and the compound was released with 3.5N NH₃ in MeOH solution. After concentration, pure product was solved in 1N HCl and lyophilised. UPLC Rt_{E}=0.61 min, [M+H]⁺=384.2, HPLC Rt_{A}=1.531 min, ¹H NMR (400 MHz, DMSO-d₆) δ ppm 4.48 (d, 2H) 5.52 (s, 2H) 7.42 (dd, 1H) 7.44-7.60 (m, 4H) 7.76 (d, 2H) 7.80-7.86 (m, 1H) 7.86-8.01 (m, 4H) 8.34 (s, 1H) 8.81 (t, 1H) 9.17 (d, 3H).

### Example 10

### 1-(4-((1H-pyrazol-1-yl)methyl)benzyl)-N-(4-carbamimidoyl benzyl)-1H-pyrazole-4-carboxamide hydrochloride

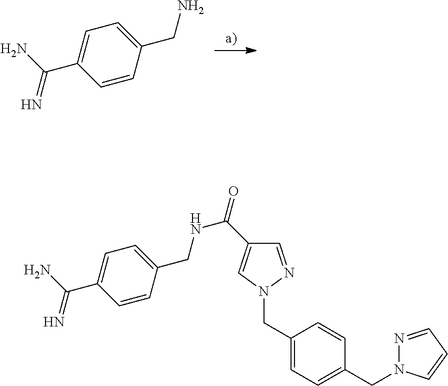 [SMILES: N=C(N)c1ccc(CN)cc1 ; N=C(N)c1ccc(CNC(=O)c2cnn(Cc3ccc(Cn4cccn4)cc3)c2)cc1]

### a) 1-(4-((1H-pyrazol-1-yl)methyl)benzyl)-N-(4-carbamimidoylbenzyl)-1H-pyrazole-4-carboxamide hydrochloride

To a solution of 1-(4-((1H-pyrazol-1-yl)methyl)benzyl)-1H-pyrazole-4-carboxylic acid (85%, 600 mg, 1.815 mmol), 4-(aminomethyl)benzamidine dihydrochloride (443 mg, 1.997 mmol) and DIPEA (1.58 ml, 9.08 mmol) in DMF (18 ml) was added HBTU (757 mg, 1.99 mmol). The reaction mixture was stirred at rt overnight, diluted with DCM and filtered through SiliaBond® Carbonate 0.7 mmol/g (Silicycle, 6.0 g, 4.2 mmol). The product was released with MeOH and concentrated. The crude product was purified by preparative HPLC (Waters Sunfire C18-OBD, 5 μm, 30×100 mm, flow: 40 mL/min, eluent: 5% to 100% ACN in H₂O in 25 min, ACN and H₂O containing 0.1% TFA). Pure HPLC fractions were concentrated, diluted with MeOH (40 ml) and HCl 2N (2 ml), sonicated and filtered. The filtrate was concentrated to remove MeOH and lyophilised to obtain the title compound. UPLC Rt_{E}=0.52 min, [M+H]⁺=414.2, HPLC Rt_{A}=1.283 min, ¹H NMR (400 MHz, DMSO-d₆) δ ppm 4.47 (d, 2H) 5.32 (d, 4H) 6.26 (t, 1H) 7.15-7.27 (m, 4H) 7.41-7.53 (m, 3H) 7.73-7.85 (m, 3H) 7.91 (s, 1H) 8.27 (s, 1H) 8.81 (t, 1H) 9.20 (d, 3H).

### Example 11

### N-((5-chloro-1H-indazol-3-yl)methyl)-1-(4-methoxybenzyl)-1H-pyrazole-4-carboxamide

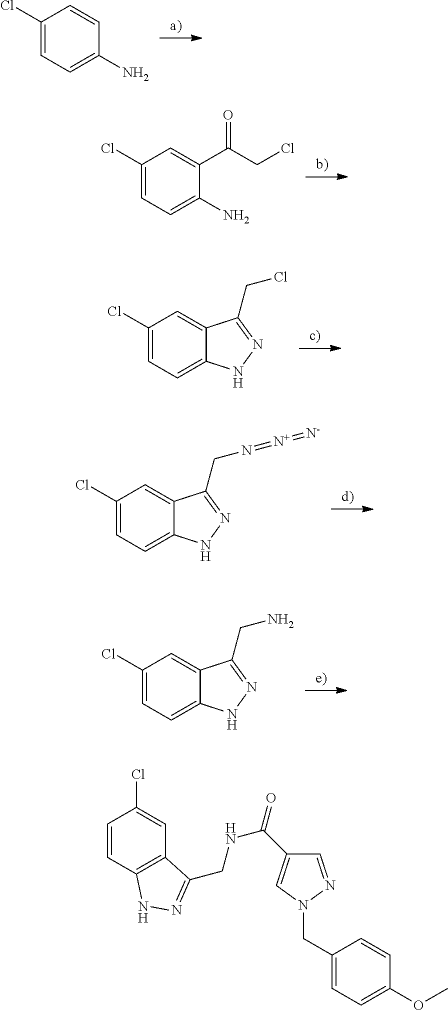 [SMILES: Nc1ccc(Cl)cc1 ; Nc1ccc(Cl)cc1C(=O)CCl ; ClCc1n[nH]c2ccc(Cl)cc12 ; [N-]=[N+]=NCc1n[nH]c2ccc(Cl)cc12 ; NCc1n[nH]c2ccc(Cl)cc12 ; COc1ccc(Cn2cc(C(=O)NCc3n[nH]c4ccc(Cl)cc34)cn2)cc1]

### a) 1-(2-amino-5-chlorophenyl)-2-chloroethanone

The title compound was prepared in a similar manner as described in J. Org. Chem., 1979, 44, 578: To a solution of Boron Trichloride 1M in toluene (43.1 ml, 43.1 mmol) was added dropwise at 120° C., a solution of 4-chloroaniline (5 g, 39.2 mmol) in toluene (43 ml). To the resulting mixture was added chloroacetonitrile (2.99 ml, 47.0 mmol) followed by aluminium trichloride (5.75 g, 43.1 mmol). The reaction mixture was stirred at 120° C. during 24 h (HCl formed was trapped by aqueous NaOH solution). After cooling to rt, the reaction mixture was quenched by a slow addition of aqueous HCl 2N (100 ml). The resulting yellow precipitate was hydrolysed by heating the mixture at 80° C. for 30 min. The cooled mixture was extracted with DCM (3×100 ml), the combined organic layers were dried (Na₂SO₄), filtered and concentrated. The title product was recrystallised from a hot EtOH/n-Hexane solution (30 ml). UPLC Rt_{E}=0.89 min, [M+H]⁺=204.1-206.1.

### b) 5-chloro-3-(chloromethyl)-1H-indazole

The title compound was prepared in a similar manner as described in J. Org. Chem., 1979, 44, 578: To a suspension of 1-(2-amino-5-chlorophenyl)-2-chloroethanone (1.35 g, 6.63 mmol) in HCl 37% in water was added dropwise at −10° C. a solution of Sodium nitrite (0.503 g, 7.29 mmol) in H₂O (3.1 ml). The reaction mixture was stirred at this temperature during 1 h, then a solution of Tin (II) chloride (3.02 g, 15.91 mmol) in HCl 37% in H₂O (9.2 ml) was added and the reaction mixture was stirred 1 h at the same temperature. Ice-water was added and the precipitate formed was filtered, washed with ice-water and dried on HV. UPLC Rt_{E}=0.89 min, [M+H]⁺=201.1-203.1.

### c) 3-(azidomethyl)-5-chloro-1H-indazole

The title compound was prepared in a similar manner as described in Synthetic Communications, 1988, 18(3), 259-264: To a solution of 5-chloro-3-(chloromethyl)-1H-indazole (1.26 g, 6.28 mmol) in DMF (12.6 ml) and H₂O (1.3 ml) was added Sodium azide (0.53 g, 8.16 mmol). The reaction mixture was stirred at 90° C. during 1 h. Volatiles were evaporated and the crude residue was diluted with ice-water (50 ml) and brine (50 ml), extracted with EtOAc (4×50 ml). The combined organic layers were dried (Na₂SO₄), filtered and concentrated. The crude residue was engaged in the next step without further purification. UPLC Rt_{E}=0.90 min, [M+H]⁺=206.2-208.3.

### d) (5-chloro-1H-indazol-3-yl)methanamine

The title compound was prepared in a similar manner as described in Synthetic Communications, 1988, 18(3), 259-264: To a suspension of 3-(azidomethyl)-5-chloro-1H-indazole (1.39 g, 6.05 mmol) in MeOH (20 ml) under N₂ atmosphere at 0° C., was added Platinium (IV) oxide (73 mg, 0.321 mmol). The reaction was placed under hydrogen atmosphere and was stirred at rt during 1.5 h. The catalyst was removed by filtration through a pad of Celite and washed with MeOH. Solvents were concentrated. The residue was taken up in aqueous HCl 1N (70 ml) and washed with MTBE (2×70 ml). The aqueous layer was brought to basic pH with 1 N NaOH and extracted with EtOAc (6×70 ml). The combined EtOAc layers were dried (Na₂SO₄), filtered and concentrated. The crude residue was engaged in the next step without further purification. UPLC Rt_{E}=0.31 min, [M+H]⁺=182.1; HPLC Rt_{A}=1.062 min.

### e) N-((5-chloro-1H-indazol-3-yl)methyl)-1-(4-methoxybenzyl)-1H-pyrazole-4-carboxamide

To resin PL-TFP, 1.4 mmol/g, 150-300 um (300 mg) was added a solution of 1-(4-methoxybenzyl)-1H-pyrazole-4-carboxylic acid (64 mg, 0.276 mmol) and 4-DMAP (20 mg, 0.164 mmol) in DCM (4 ml)/DMF (1 ml). After 10 min shaking at rt, N,N′-diisopropylcarbodiimide (0.172 ml, 1.102 mmol) was added and the mixture was shaked at rt overnight. The resin (loaded with the activated ester) was filtered and washed with DMF (2 ml), DCM (10 ml) and THF (10 ml). The wet resin was suspended in THF (3 ml), then pipetted back to flask. A solution of the amine 5-chloro-1H-indazol-3-yl)methanamine (50 mg, 0.275 mmol) and TEA (230 μL, 1.653 mmol) in THF (2 ml) was added to the resin suspension and the mixture was shaked at rt for 4 days, filtered and washed with THF. The filtrate was diluted with EtOAc and washed with HCl 1N, aq. sat. NaHCO₃ and brine. The organic layer was dried (Na₂SO₄), filtered and concentrated. The residue was triturated with ACN (1 ml), the precipitate was filtered, washed with ACN (1 ml) and dried on HV.UPLC Rt_{E}=0.85 min, [M+H]⁺=396.3-398.4, ¹H NMR (400 MHz, DMSO-d₆) δ ppm 3.71 (s, 3H) 4.68 (d, 2H) 5.23 (s, 2H) 6.70-6.99 (m, 2H) 7.10-7.26 (m, 2H) 7.31 (dd, 1H) 7.50 (d, 1H) 7.81-7.93 (m, 2H) 8.20 (s, 1H) 8.65 (br. s., 1H) 13.01 (s, 1H).

### Example 12

### N-((5-chloro-1H-indol-3-yl)methyl)-1-((2-methylquinolin-6-yl)methyl)-1H-pyrazole-4-carboxamide

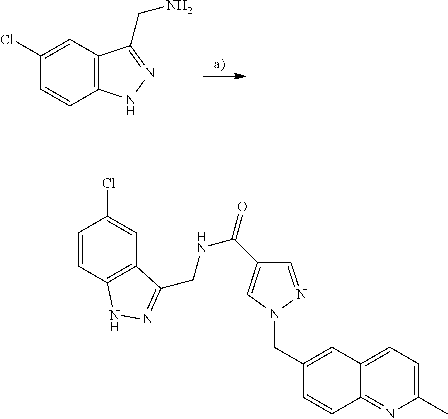 [SMILES: NCc1n[nH]c2ccc(Cl)cc12 ; Cc1ccc2cc(Cn3cc(C(=O)NCc4n[nH]c5ccc(Cl)cc45)cn3)ccc2n1]

### a) N-((5-chloro-1H-indol-3-yl)methyl)-1-((2-methylquinolin-6-yl)methyl)-1H-pyrazole-4-carboxamide

1-((2-methylquinolin-6-yl)methyl)-1H-pyrazole-4-carboxylic acid (150 mg, 0.281 mmol), HBTU (160 mg, 0.421 mmol) and DIPEA (0.098 ml, 0.561 mmol) were solved in DMF (2 ml) and stirred at rt during 1 h. Then (5-chloro-1H-indol-3-yl)methanamine (76 mg, 0.421 mmol) was added and the reaction mixture was stirred at rt overnight, diluted with EtOAc and washed with H₂O then with aq. sat. NaHCO₃. The organic layer was dried (Na₂SO₄), filtered and concentrated. The crude residue was purified by flash chromatography on silica gel (c-hexane/EtOAc 0/10 to 0/10 then DCM to DCM/MeOH 9/1). Right fractions were concentrated and purified by preparative HPLC (Waters Sunfire C18-OBD, 5 μm, 30×100 mm, flow: 40 mL/min, eluent: 5% to 100% ACN in H₂O in 25 min, ACN and H₂O containing 0.1% TFA). Pure HPLC fractions were neutralized with aq. sat. NaHCO₃ and extracted with DCM, dried (Na₂SO₄), filtered and concentrated to give the title product. UPLC Rt_{E}=0.63 min, [M+H]⁺=430.4, HPLC Rt_{A}=1.502 min, ¹H NMR (400 MHz, DMSO-d₆) δ ppm 2.65 (s, 3H) 4.51 (d, 2H) 5.52 (s, 2H) 7.07 (dd, 1H) 7.31-7.44 (m, 3H) 7.58 (dd, 1H) 7.67 (d, 1H) 7.75-7.79 (m, 1H) 7.87-7.95 (m, 2H) 8.23 (d, 1H) 8.32 (s, 1H) 8.41 (br. s., 1H) 11.11 (br. s., 1H).

### Example 13

### N-((5-amino-3-methylpyrazin-2-yl)methyl)-1-((2-methylquinolin-6-yl)methyl)-1H-1,2,3-triazole-4-carboxamide

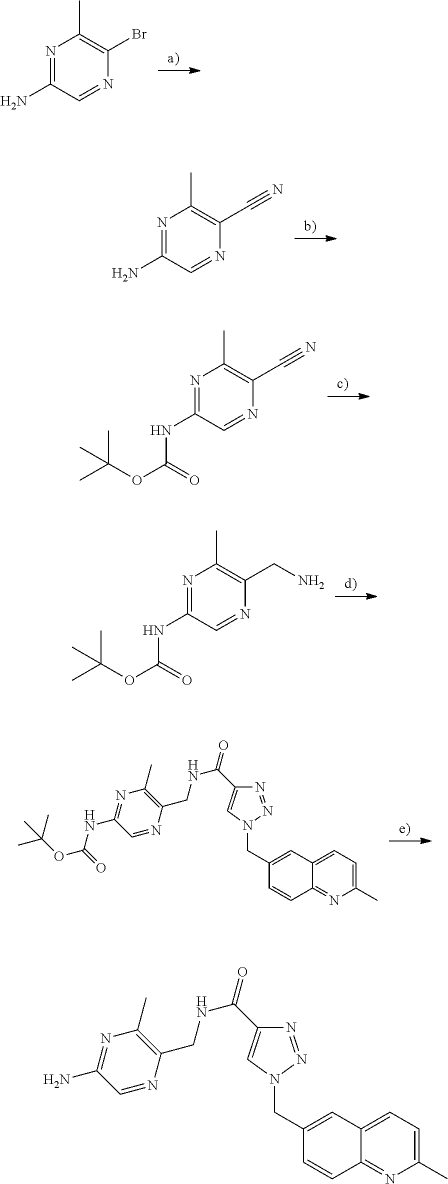

### a) 5-amino-3-methylpyrazine-2-carbonitrile

A suspension of 5-bromo-6-methylpyrazin-2-amine (500 mg, 2.66 mmol), sodium cyanide (1303 mg, 26.6 mmol), copper(I) cyanide (2382 mg, 26.6 mmol) in DMF (10 ml) was stirred at 120° C. during 15 h. After cooling to rt, the reaction mixture was diluted with H₂O (10 ml) and concentrated. The residue was partitioned between EtOAc (100 ml) and 3% ammonium hydroxide (150 ml). The aqueous layer was extracted further with EtOAc (2×70 ml), and the combined organic layers were washed again with 3% ammonium hydroxide (50 ml), brine (50 ml), dried (Na₂SO₄), filtered and concentrated to give the title compound. [M+H]⁺=135.0, HPLC Rt_{A}=0.75 min.

### b) tert-butyl 5-cyano-6-methylpyrazin-2-ylcarbamate

The title compound was prepared in a similar manner as described in J. Med. Chem., 1998, 41, 4466: A mixture of 5-amino-3-methylpyrazine-2-carbonitrile (317 mg, 2.056 mmol), Boc₂O (0.492 ml, 2.118 mmol), DMAP (25.1 mg, 0.206 mmol), and TEA (0.315 ml, 2.262 mmol) in DCM (5.5 ml) was stirred at rt for 1 day. Another 1 eq of Boc₂O (0.478 ml, 2.056 mmol), 1 eq of TEA (0.286 ml, 2.056 mmol), 0.1 eq of DMAP (25.1 mg, 0.206 mmol) and 5 ml of DCM were added, and the stirring continued for 1.5 h at rt.

The reaction was quenched with 1N HCl (5 ml), and the layers were separated. The aqueous layer was further extracted with DCM (2×20 ml), the combined organic layers were washed with 1N NaOH aqueous solution (10 ml), dried (Na₂SO₄), filtered and concentrated to give the bis-boc product. This was solved in MeOH (8.5 ml) and DCM (2.5 ml) then 1N NaOH (3.08 ml, 3.08 mmol) and 30% H₂O₂ (0.095 ml, 3.08 mmol) were added. The reaction mixture was stirred at rt for 4 h. The reaction was quenched with 10% Na₂S₂O₃ solution (2 ml) and concentrated. The crude residue was solved in EtOAc (10 ml) and washed with H₂O (10 ml). The aqueous layer was extracted with EtOAc (2×20 ml). The combined organic layers were washed with brine (10 ml), dried (Na₂SO₄), filtered and concentrated. The crude residue was purified by flash chromatography on silica gel (c-hexane/EtOAc 10/0 to 85/15) to yield the title product. UPLC Rt_{C}=1.07 min, [M+H]⁺=235.4, HPLC Rt_{A}=2.045 min.

### c) tert-butyl 5-(aminomethyl)-6-methylpyrazin-2-ylcarbamate

To a solution of tert-butyl 5-cyano-6-methylpyrazin-2-ylcarbamate (278 mg, 1.187 mmol) in 2M NH₃ in MeOH (30 ml) was added Pd/C 10% (200 mg, 0.19 mmol). The reaction was placed under hydrogen atmosphere (3 bar) and was stirred at rt during 8 h. The catalyst was removed by filtration through a pad of Celite and washed with MeOH. Solvents were concentrated to give the title compound. UPLC Rt_{C}=0.59 min, [M+H]⁺=239.3, HPLC Rt_{A}=1.19 min.

### d) tert-butyl 6-methyl-5-((1-((2-methylquinolin-6-yl)methyl)-1H-1,2,3-triazole-4-carboxamido)methyl)pyrazin-2-ylcarbamate

To a solution of 1-((2-methylquinolin-6-yl)methyl)-1H-1,2,3-triazole-4-carboxylic acid (64%, 50 mg, 0.119 mmol), EDC.HCl (29.7 mg, 0.155 mmol) and HOAT (21.11 mg, 0.155 mmol) in DMF (1 ml) was added N-Methylmorpholine (0.039 ml, 0.358 mmol). The reaction mixture was stirred at rt for 1 h then tert-butyl 5-(aminomethyl)-6-methylpyrazin-2-ylcarbamate (80%, 35.5 mg, 0.119 mmol) was added and the reaction mixture was stirred 3 h at rt, concentrated, treated with MeOH (0.5 ml) and ACN (0.5 ml) and filtered. The filtrate was purified by preparative HPLC (Waters Sunfire C18-OBD, 5 μm, 30×100 mm, flow: 40 mL/min, eluent: 5% to 100% ACN in H₂O in 25 min, ACN and H₂O containing 0.1% TFA). UPLC Rt_{C}=0.89 min, [M+H]⁺=489.5.

### e) N-((5-amino-3-methylpyrazin-2-yl)methyl)-1-((2-methylquinolin-6-yl)methyl)-1H-1,2,3-triazole-4-carboxamide

tert-butyl 6-methyl-5-((1-((2-methylquinolin-6-yl)methyl)-1H-1,2,3-triazole-4-carboxamido)methyl)pyrazin-2-ylcarbamate (36 mg, 0.074 mmol) was solved in DCM (1 ml) then TFA (0.28 ml) was added and the reaction mixture was stirred at rt overnight, concentrated, diluted in MeOH, subjected on PL-HCO3 MP-Resin column to obtain after concentration of the filtrate, the title compound. UPLC Rt_{C}=0.52 min, [M+H]⁺=389.4, HPLC Rt_{G}=2.42 min, ¹H NMR (400 MHz, DMSO-d₆) δ ppm 2.30 (s, 3H) 2.64 (s, 3H) 4.41 (d, 2H) 5.83 (s, 2H) 6.17 (br. s., 2H) 7.43 (d, 1H) 7.60-7.70 (m, 2H) 7.85 (d, 1H) 7.91 (d, 1H) 8.24 (d, 1H) 8.59 (d, 1H) 8.71 (s, 1H).

### Example 14

### N-(6-chloro-2-fluoro-3-methoxybenzyl)-1-((2-methylquinolin-6-yl)methyl)-1H-1,2,3-triazole-4-carboxamide

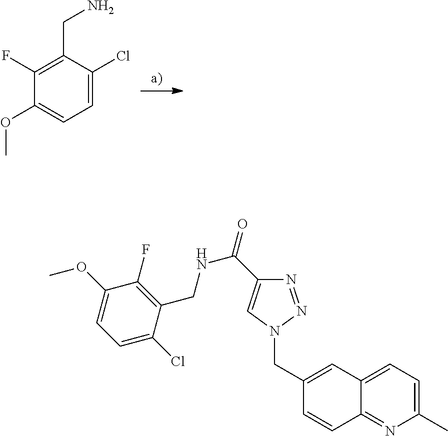

### a) N-(6-chloro-2-fluoro-3-methoxybenzyl)-1-((2-methylquinolin-6-yl)methyl)-1H-1,2,3-triazole-4-carboxamide

To a suspension of 1-((2-methylquinolin-6-yl)methyl)-1H-1,2,3-triazole-4-carboxylic acid (75 mg, 0.280 mmol), (6-chloro-2-fluoro-3-methoxyphenyl)methanamine (58.3 mg, 0.308 mmol) and DIPEA (0.098 ml, 0.559 mmol) in DCM (2.8 ml) was added propylphosphonic anhydride 50% in EtOAc (0.208 ml, 0.349 mmol). The reaction mixture was stirred at rt for 4 h. NaOH 0.1M was added to the reaction mixture and extracted with DCM (3×10 ml). The combined organic layers were washed with brine, dried (Na₂SO₄), filtered and concentrated. The crude residue was purified by flash chromatography on silica gel (DCM/MeOH 10/0 to 9/1). After concentration, the compound was triturated in Et₂O (4 ml), the precipitate was filtered and dried on HV. UPLC Rt_{C}=0.88 min, [M+H]⁺=440.2-442.2, HPLC Rt_{G}=1.332 min, ¹H NMR (400 MHz, DMSO-d₆) δ ppm 2.66 (s, 3H) 3.84 (s, 3H) 4.57 (d, 2H) 5.83 (s, 2H) 7.15 (t, 1H) 7.20-7.31 (m, 1H) 7.44 (d, 1H) 7.66 (dd, 1H) 7.86 (d, 1H) 7.92 (d, 1H) 8.25 (d, 1H) 8.69 (s, 2H).

### Examples 15 to 28

The compounds of the following tabulated Examples were prepared in analogy to the methods described for Examples 1 to 14 using the appropriate amines and carboxylic acids.

(HPLC-parameters are those of method G or I as indicated by Rt_{G} or Rt_{I} respectively described in the general part of the experimental Section).

| | | | HPLC- |
|---|---|---|---|
| Ex. | | LRMS | retention |
| No. | Structure and Name | m/z = | time (min) |
| 15 | 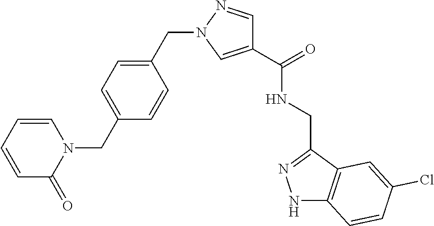 [SMILES: O=C(NCc1n[nH]c2ccc(Cl)cc12)c1cnn(Cc2ccc(Cn3ccccc3=O)cc2)c1] N-((5-chloro-1H-indazol-3-yl)methyl)-1-(4- ((2-oxopyridin-1(2H)-yl)methyl)benzyl)-1H- pyrazole-4-carboxamide | 473.4 [M + H]⁺ | Rt_{G} = 2.97 |
| 16 | 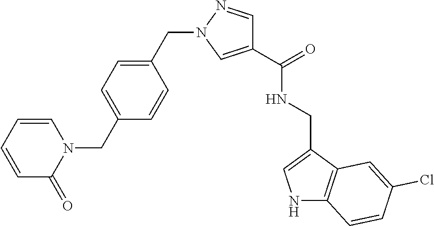 [SMILES: O=C(NCc1c[nH]c2ccc(Cl)cc12)c1cnn(Cc2ccc(Cn3ccccc3=O)cc2)c1] N-((5-chloro-1H-indol-3-yl)methyl)-1-(4-((2- oxopyridin-1(2H)-yl)methyl)benzyl)-1H- pyrazole-4-carboxamide | 472.4 [M + H]⁺ | Rt_{G} = 3.15 |
| 17 | 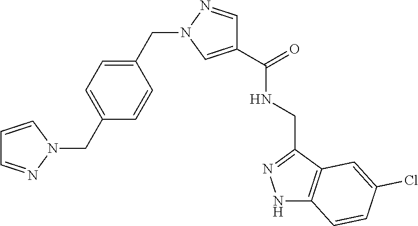 [SMILES: O=C(NCc1n[nH]c2ccc(Cl)cc12)c1cnn(Cc2ccc(Cn3cccn3)cc2)c1] 1-(4-((1H-pyrazol-1-yl)methyl)benzyl)-N- ((5-chloro-1H-indazol-3-yl)methyl)-1H- pyrazole-4-carboxamide | 446.4 [M + H]⁺ | Rt_{G} = 3.11 |
| 18 | 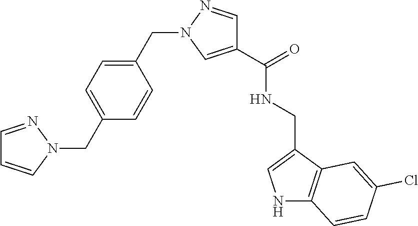 [SMILES: O=C(NCc1c[nH]c2ccc(Cl)cc12)c1cnn(Cc2ccc(Cn3cccn3)cc2)c1] 1-(4-((1H-pyrazol-1-yl)methyl)benzyl)-N- ((5-chloro-1H-indol-3-yl)methyl)-1H- pyrazole-4-carboxamide | 445.4 [M + H]⁺ | Rt_{G} = 3.27 |
| 19 | 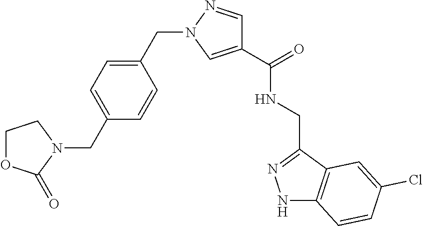 [SMILES: O=C(NCc1n[nH]c2ccc(Cl)cc12)c1cnn(Cc2ccc(CN3CCOC3=O)cc2)c1] N-((5-chloro-1H-indazol-3-yl)methyl)-1-(4- ((2-oxooxazolidin-3-yl)methyl)benzyl)-1H- pyrazole-4-carboxamide | 465.5 [M + H]⁺ | Rt_{G} = 2.96 |
| 20 | 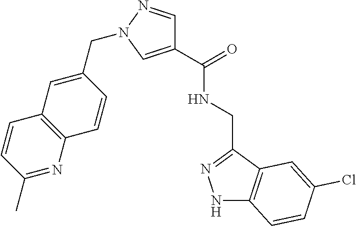 [SMILES: Cc1ccc2cc(Cn3cc(C(=O)NCc4n[nH]c5ccc(Cl)cc45)cn3)ccc2n1] N-((5-chloro-1H-indazol-3-yl)methyl)-1-((2- methylquinolin-6-yl)methyl)-1H-pyrazole-4- carboxamide | 431.4 [M + H]⁺ | Rt_{G} = 2.56 |
| 21 | 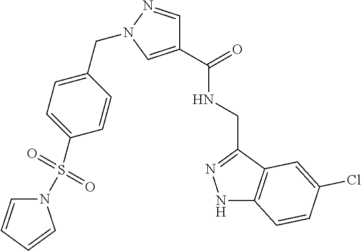 [SMILES: O=C(NCc1n[nH]c2ccc(Cl)cc12)c1cnn(Cc2ccc(S(=O)(=O)n3cccc3)cc2)c1] 1-(4-((1H-pyrrol-1-yl)sulfonyl)benzyl)-N- ((5-chloro-1H-indazol-3-yl)methyl)-1H- pyrazole-4-carboxamide | 495.4 [M + H]⁺ | Rt_{G} = 3.46 |
| 22 | 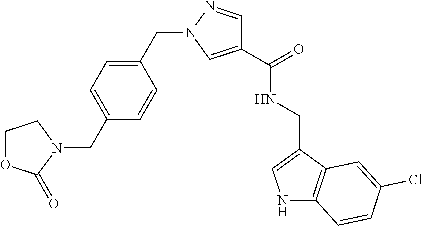 [SMILES: O=C(NCc1c[nH]c2ccc(Cl)cc12)c1cnn(Cc2ccc(CN3CCOC3=O)cc2)c1] N-((5-chloro-1H-indol-3-yl)methyl)-1-(4-((2- oxooxazolidin-3-yl)methyl)benzyl)-1H- pyrazole-4-carboxamide | 464.5 [M + H]⁺ | Rt_{G} = 3.17 |
| 23 | 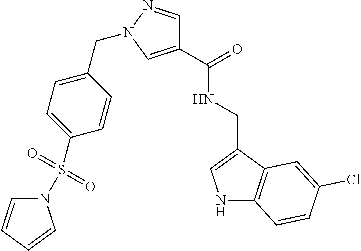 [SMILES: O=C(NCc1c[nH]c2ccc(Cl)cc12)c1cnn(Cc2ccc(S(=O)(=O)n3cccc3)cc2)c1] 1-(4-((1H-pyrrol-1-yl)sulfonyl)benzyl)-N- ((5-chloro-1H-indol-3-yl)methyl)-1H- pyrazole-4-carboxamide | 494.4 [M + H]⁺ | Rt_{G} = 3.63 |
| 24 | 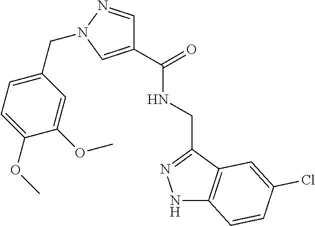 [SMILES: COc1ccc(Cn2cc(C(=O)NCc3n[nH]c4ccc(Cl)cc34)cn2)cc1OC] N-((5-chloro-1H-indazol-3-yl)methyl)-1- (3,4-dimethoxybenzyl)-1H-pyrazole-4- carboxamide | 426.4 [M + H]⁺ | Rt_{I} = 0.80 |
| 25 | 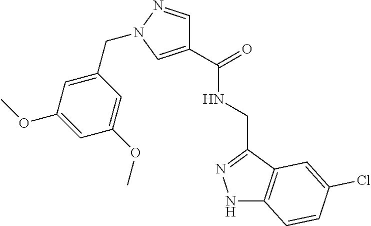 [SMILES: COc1cc(Cn2cc(C(=O)NCc3n[nH]c4ccc(Cl)cc34)cn2)cc(OC)c1] N-((5-chloro-1H-indazol-3-yl)methyl)-1- (3,5-dimethoxybenzyl)-1H-pyrazole-4- carboxamide | 426.4 [M + H]⁺ | Rt_{I} = 0.88 |
| 26 | 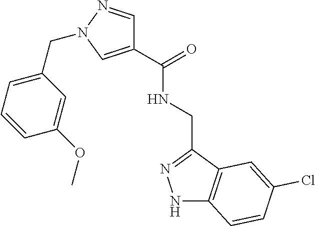 [SMILES: COc1cccc(Cn2cc(C(=O)NCc3n[nH]c4ccc(Cl)cc34)cn2)c1] N-((5-chloro-1H-indazol-3-yl)methyl)-1-(3- methoxybenzyl)-1H-pyrazole-4- carboxamide | 396.3 [M + H]+ | Rt_{I} = 0.86 |
| 27 | 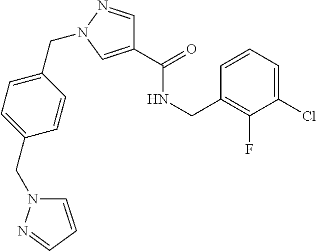 [SMILES: O=C(NCc1cccc(Cl)c1F)c1cnn(Cc2ccc(Cn3cccn3)cc2)c1] 1-(4-((1H-pyrazol-1-yl)methyl)benzyl)-N- (3-chloro-2-fluorobenzyl)-1H-pyrazole-4- carboxamide | 424.3 [M + H]⁺ | Rt_{G} = 3.34 |
| 28 | 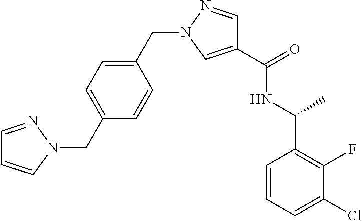 [SMILES: C[C@@H](NC(=O)c1cnn(Cc2ccc(Cn3cccn3)cc2)c1)c1cccc(Cl)c1F] (R)-1-(4-((1H-pyrazol-1-yl)methyl)benzyl)- N-(1-(3-chloro-2-fluorophenyl)ethyl)-1H- pyrazole-4-carboxamide | 438.4 [M + H]⁺ | Rt_{G} = 3.46 |

### Example 29

### In Vitro Inhibition of Plasma Kallikrein

### Materials

The fluorogenic substrate _{D}Pro-Phe-Arg-(Rh110)-γGlu-OH (where _{D}Pro is the amino acid d-proline, Rh110 is the fluorophore rhodamine 110 and γGlu is a glutamine linked to Rh110 via the gamma-carbonyl function; from Biosyntan, Berlin, Germany), being based on the chromogenic substrate described in Gallimore et al (Thromb Res 25, 293-298, 1982), was dissolved in DMSO at 5 mM and stored at −80° C. All other chemicals were of analytical grade.

Human plasma kallikrein was purchased from Kordia (Leiden, Netherlands, batch HPKA 1303) in lyophilized form.

The protein solution was reconstituted from the lyophilisate by addition of deinonized water. The resulting stock solution comprised the protein at a concentration of 0.17 mg/ml in 4 mM sodium acetate/HCl and 150 mM NaCl at pH 5.3. The stock solution was stored in aliquots at −80° C. Enzymatic reactions were conducted in ‘assay buffer’, comprising 50 mM Hepes/NaOH at pH 7.8, 150 mM NaCl, 1 mM EDTA and 0.05% (w/v) CHAPS.

Both, enzyme and substrate were diluted in assay buffer.

All protein and peptide containing solutions were handled in siliconized tubes (Life Systems Design, Merenschwand, Switzerland). The compound solutions as well as the enzyme and the substrate solutions were transferred to 384-well plates (black Cliniplate; cat. no. 95040020 Labsystems Oy, Finland) by means of a CyBi-Well 96-channel pipettor (CyBio AG, Jena, Germany). Plate measurements were conducted by the means of a Safire2 reader (TECAN, Maennedorf, Switzerland). The Safire2 is a monochomator-based instrument and wavelengths of 485 nm and 535 nm were taken for fluorescence excitation and emission acquisition, respectively. The bandwidths were set to 10 nm in both the excitation and the emission path. The fluorescence in each well was excited by three flashes per measurement.

### Determination of IC₅₀ Values

For the determination of IC₅₀ values, the assays were performed at room temperature in 384-well plates with a total assay volume of 25.25 μl per well.

The test compound was dissolved in 90% (v/v) DMSO/water. For the assays, 250 mL of the 90% (v/v) DMSO/water solution or compound solution were added per well, followed by the addition of 12.5 μl protease solution (protease in assay buffer). The final assay concentration of the human plasma kallikrein was nominally 12 pM, the 11 compound concentrations in the dilution series were in the range form 1 nM to 100 μM. After 1 hour of pre-incubation at room temperature, the reactions were started by the addition of 12.5 μl substrate solution (in assay buffer, final assay concentration was 0.5 μM). After the addition of the substrate solution, the final DMSO concentration in the assay was 0.9% (v/v). The effect of the compound on the enzymatic activity was obtained from the linear part of the progress curves and determined after 1 hour (t=60 min). The IC₅₀ value was calculated from the plot of percentage of inhibition vs. inhibitor concentration by a logistics fit according to the following equation:

y=A2+(A1−A2)/(1+(x/IC50)̂p)

where y is the %-inhibition at the inhibitor concentration, x. A1 is the lowest inhibition value, i.e. 0%, and A2 the maximum inhibition value, i.e. 100%. The exponent, p, is the Hill coefficient. The curve fitting was conducted with the non-linear regression routine of the analysis software Origin 7.5SR6 (Origin Lab Corporation).

| Example | |
|---|---|
| number | IC50 (μM) |
| Example 1 | 10.238 |
| Example 2 | 3.60075 |
| Example 3 | 0.57339 |
| Example 4 | 2.4215 |
| Example 5 | 2.9184 |
| Example 6 | 2.8351 |
| Example 7 | 1.311 |
| Example 8 | 1.61655 |
| Example 9 | 0.0002918 |
| Example 10 | 0.0008779 |
| Example 11 | 0.353095 |
| Example 12 | 0.0243255 |
| Example 13 | 0.78353 |
| Example 14 | 0.042 |
| Example 15 | 0.0001067 |
| Example 16 | 0.0003275 |
| Example 17 | 0.0005544 |
| Example 18 | 0.0043854 |
| Example 19 | 0.0090992 |
| Example 20 | 0.0108635 |
| Example 21 | 0.0463575 |
| Example 22 | 0.059976 |
| Example 23 | 0.133735 |
| Example 24 | 0.28482 |
| Example 25 | 0.311735 |
| Example 26 | 0.429355 |
| Example 27 | 0.844345 |
| Example 28 | 27.1535 |

The compounds N-((2-chloro-6-methylpyridin-3-yl)methyl)-1-((2-methylquinolin-6-yl)methyl)-1H-1,2,3-triazole-4-carboxamide, N-((6-methoxy-2,4-dimethylpyridin-3-yl)methyl)-1-((2-methylquinolin-6-yl)methyl)-1H-1,2,3-triazole-4-carboxamide, N-(4-chlorobenzyl)-1-(4-methoxybenzyl)-1H-pyrazole-4-carboxamide, N-(4-chlorobenzyl)-1-(3-methoxybenzyl)-1H-pyrazole-4-carboxamide and N-(3-chlorobenzyl)-1-((1-methyl-1H-benzo[d][1,2,3]triazol-5-yl)methyl)-1H-pyrazole-4-carboxamide exhibit efficacy in the above-described assay with an IC₅₀>30 μM.

The following are further embodiments of the invention:

### Embodiment 1

### A Compound of Formula (I) in Free Form or in Pharmaceutically Acceptable Salt Form

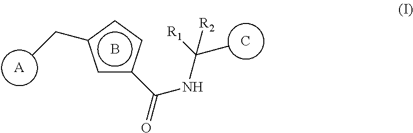

wherein

R₁ and R₂ are each independently hydrogen or methyl;

A is a 5- to 10-membered monocyclic or fused polycyclic aromatic ring system, which may contain 1, 2, 3, or 4 heteroatoms selected from N, O and S, wherein the ring system

A is unsubstituted or substituted once, twice or three times by R₃;

wherein A is neither unsubstituted phenyl nor unsubstituted pyridinyl;

each R₃ is independently selected from halogen, C₁-C₄alkyl, C₁-C₄alkoxy, oxo, cyano, C₁-C₄halogenalkyl, NR₄R₅; or

R₃ is a 5- to 6-membered monocyclic ring system which may be aromatic, saturated or unsaturated non-aromatic and which may contain 1, 2, 3 or 4 heteroatoms selected from N, O and S, wherein the ring system R₃ is attached to A via bond, C₁-C₂alkylene or SO₂,

wherein the ring system R₃ is in turn optionally substituted with oxo;

R₄ and R₅ are independently selected from hydrogen or C₁-C₄alkyl;

B is a five-membered monocyclic aromatic ring system which contains 1, 2, 3, or 4 heteroatoms selected from N, O and S;

C is a 5- to 10-membered monocyclic or fused polycyclic aromatic ring system, which may contain 1, 2, 3, or 4 heteroatoms selected from N, O and S, wherein the ring system C is unsubstituted or substituted once, twice or three times by R₆;

each R₆ is independently selected from halogen, C₁-C₄alkyl, C₁-C₄alkoxy, C₁-C₄halogenalkyl, amino, aminoC₁-C₄alkyl, cyano, C₂-C₄alkynyl;

wherein C is neither a 2-aminopyridinyl nor a 6-aminopyridinyl residue, for use as a medicament.

### Embodiment 2

### A Compound of Formula (I) for Use as a Medicament According to Embodiment 1 in Free Form or in Pharmaceutically Acceptable Salt orm wherein

R₁ and R₂ are hydrogen; or

R₁ is methyl and R₂ is hydrogen.

### Embodiment 3

### A Compound of Formula (I) for Use as a Medicament According to Embodiment 1 or 2 in Free Form or in Pharmaceutically Acceptable Salt Form wherein

A is selected from phenyl, naphthyl, pyridinyl or quinolinyl.

### Embodiment 4

### A Compound of Formula (I) for Use as a Medicament According to any of the Preceding Embodiments in Free Form or in Pharmaceutically Acceptable Salt Form wherein

each R₃ independently is methyl, methoxy or

R₃ is pyridinyl, oxazolidinyl, pyrazolyl, pyrrolyl attached to A via CH₂— or SO₂—, wherein

R₃ is optionally substituted with oxo.

### Embodiment 5

### A Compound of Formula (I) for Use as a Medicament According to any of the Preceding Embodiments in Free Form or in Pharmaceutically Acceptable Salt Form wherein

B is selected from

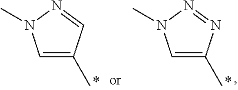

wherein the bond marked with * is attached to the carboxamide group.

### Embodiment 6

### A Compound of Formula (I) for Use as a Medicament According to any of the Preceding Embodiments in Free Form or in Pharmaceutically Acceptable Salt Form wherein

C is phenyl, pyridinyl, pyrrolopyridinyl, indolyl, indazolyl, isoquinolinyl or naphthyl.

### Embodiment 7

### A Compound of Formula (I) for Use as a Medicament According to any of the Preceding Embodiments in Free Form or in Pharmaceutically Acceptable Salt Form wherein

each R₆ is independently selected from chloro, fluoro, methyl, methoxy and trifluoromethyl.

### Embodiment 8

### A Compound of Formula (I) in Free Form or in Pharmaceutically Acceptable Salt Form which is Selected From

N-((2,4-dimethylpyridin-3-yl)methyl)-1-((2-methylquinolin-6-yl)methyl)-1H-1,2,3-triazole-4-carboxamide;
N-((4-methoxy-2-methylpyridin-3-yl)methyl)-1-((2-methylquinolin-6-yl)methyl)-1H-1,2,3-triazole-4-carboxamide;
N-((6-hydroxy-2,4-dimethylpyridin-3-yl)methyl)-1-((2-methylquinolin-6-yl)methyl)-1H-1,2,3-triazole-4-carboxamide;
N-((2-hydroxy-3,5-dimethylpyridin-4-yl)methyl)-1-((2-methylquinolin-6-yl)methyl)-1H-1,2,3-triazole-4-carboxamide;
N-((5-methyl-1H-pyrrolo[2,3-b]pyridin-3-yl)methyl)-1-((2-methylquinolin-6-yl)methyl)-1H-1,2,3-triazole-4-carboxamide;
N-((4-methyl-2-(trifluoromethyl)pyridin-3-yl)methyl)-1-((2-methylquinolin-6-yl)methyl)-1H-1,2,3-triazole-4-carboxamide;
1-(4-((1H-pyrazol-1-yl)methyl)benzyl)-N-(3-chlorobenzyl)-1H-pyrazole-4-carboxamide;
(1-(4-((1H-pyrazol-1-yl)methyl)benzyl)-N-(1-(3-chlorophenyl)ethyl)-1H-pyrazole-4-carboxamide;
N-((5-chloro-1H-indazol-3-yl)methyl)-1-(4-methoxybenzyl)-1H-pyrazole-4-carboxamide;
N-((5-chloro-1H-indol-3-yl)methyl)-1-((2-methylquinolin-6-yl)methyl)-1H-pyrazole-4-carboxamide;
N-((5-amino-3-methylpyrazin-2-yl)methyl)-1-((2-methylquinolin-6-yl)methyl)-1H-1,2,3-triazole-4-carboxamide;
N-(6-chloro-2-fluoro-3-methoxybenzyl)-1-((2-methylquinolin-6-yl)methyl)-1H-1,2,3-triazole-4-carboxamide;
N-((5-chloro-1H-indazol-3-yl)methyl)-1-(4-((2-oxopyridin-1(2H)-yl)methyl)benzyl)-1H-pyrazole-4-carboxamide;
N-((5-chloro-1H-indol-3-yl)methyl)-1-(4-((2-oxopyridin-1 (2H)-yl)methyl)benzyl)-1H-pyrazole-4-carboxamide;
1-(4-((1H-pyrazol-1-yl)methyl)benzyl)-N-((5-chloro-1H-indazol-3-yl)methyl)-1H-pyrazole-4-carboxamide;
1-(4-((1H-pyrazol-1-yl)methyl)benzyl)-N-((5-chloro-1H-indol-3-yl)methyl)-1H-pyrazole-4-carboxamide;
N-((5-chloro-1H-indazol-3-yl)methyl)-1-(4-((2-oxooxazolidin-3-yl)methyl)benzyl)-1H-pyrazole-4-carboxamide;
N-((5-chloro-1H-indazol-3-yl)methyl)-1-((2-methylquinolin-6-yl)methyl)-1H-pyrazole-4-carboxamide;
1-(4-((1H-pyrrol-1-yl)sulfonyl)benzyl)-N-((5-chloro-1H-indazol-3-yl)methyl)-1H-pyrazole-4-carboxamide;
N-((5-chloro-1H-indol-3-yl)methyl)-1-(4-((2-oxooxazolidin-3-yl)methyl)benzyl)-1H-pyrazole-4-carboxamide;
1-(4-((1H-pyrrol-1-yl)sulfonyl)benzyl)-N-((5-chloro-1H-indol-3-yl)methyl)-1H-pyrazole-4-carboxamide;
N-((5-chloro-1H-indazol-3-yl)methyl)-1-(3,4-dimethoxybenzyl)-1H-pyrazole-4-carboxamide;
N-((5-chloro-1H-indazol-3-yl)methyl)-1-(3,5-dimethoxybenzyl)-1H-pyrazole-4-carboxamide;
N-((5-chloro-1H-indazol-3-yl)methyl)-1-(3-methoxybenzyl)-1H-pyrazole-4-carboxamide;
1-(4-((1H-pyrazol-1-yl)methyl)benzyl)-N-(3-chloro-2-fluorobenzyl)-1H-pyrazole-4-carboxamide; and
1-(4-((1H-pyrazol-1-yl)methyl)benzyl)-N-(1-(3-chloro-2-fluorophenyl)ethyl)-1H-pyrazole-4-carboxamide.

### Embodiment 9

### A Compound which is Selected from

N-(4-carbamimidoylbenzyl)-1-(naphthalen-2-ylmethyl)-1H-pyrazole-4-carboxamide hydrochloride; and
1-(4-((1H-pyrazol-1-yl)methyl)benzyl)-N-(4-carbamimidoylbenzyl)-1H-pyrazole-4-carboxamide hydrochloride.

### Embodiment 10

A pharmaceutical composition comprising a therapeutically effective amount of a compound according to any of embodiments 1 to 9 in free form or in pharmaceutically acceptable salt form and one or more pharmaceutically acceptable carriers.

### Embodiment 11

A combination comprising a therapeutically effective amount of the compound according to any of embodiments 1 to 9 in free form or in pharmaceutically acceptable salt form and one or more therapeutically active agents.

### Embodiment 12

A method of inhibiting plasmakallikrein activity in a subject, wherein the method comprises administering to the subject a therapeutically effective amount of the compound according to any one of embodiments 1 to 9 in free form or in pharmaceutically acceptable salt form.

### Embodiment 13

A method of treating a disorder or a disease in a subject mediated by plasmakallikrein, wherein the method comprises administering to the subject a therapeutically effective amount of the compound according to any one of embodiments 1 to 9 in free form or in pharmaceutically acceptable salt form.

### Embodiment 14

Use of a compound according to any one of embodiments 1 to 9 in free form or in pharmaceutically acceptable salt form, for the treatment of a disorder or disease in a subject mediated by plasmakallikrein.

### Embodiment 15

Use of a compound according to any one of embodiments 1 to 9 in free form or in pharmaceutically acceptable salt form, for the treatment of a disorder or disease in a subject characterized by an abnormal activity of plasmakallikrein.

1. A method of treating a disorder or a disease in a subject mediated by plasmakallikrein, wherein the method comprises administering to the subject a therapeutically effective amount of a compound of formula (I) in free form or in pharmaceutically acceptable salt form 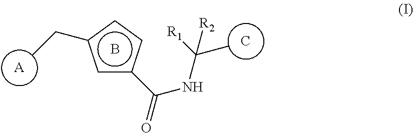
wherein
R₁ and R₂ are each independently hydrogen or methyl;
A is a 5- to 10-membered monocyclic or fused polycyclic aromatic ring system, which may contain 1, 2, 3, or 4 heteroatoms selected from N, O and S, wherein the ring system A is unsubstituted or substituted once, twice or three times by R₃;
wherein A is neither unsubstituted phenyl nor unsubstituted pyridinyl;
each R₃ is independently selected from halogen, C₁-C₄alkyl, C₁-C₄alkoxy, oxo, cyano, C₁-C₄halogenalkyl, NR₄R₅; or
R₃ is a 5- to 6-membered monocyclic ring system which may be aromatic, saturated or unsaturated non-aromatic and which may contain 1, 2, 3 or 4 heteroatoms selected from N, O and S, wherein the ring system R₃ is attached to A via bond, C₁-C₂alkylene or SO₂, wherein the ring system R₃ is in turn optionally substituted with oxo;
R₄ and R₅ are independently selected from hydrogen or C₁-C₄alkyl;
B is selected from: 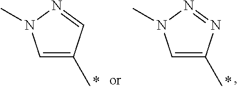
wherein the bond marked with * is attached to the carboxamide group
C is a 5- to 10-membered monocyclic or fused polycyclic aromatic ring system, which may contain 1, 2, 3, or 4 heteroatoms selected from N, O and S, wherein the ring system C is unsubstituted or substituted once, twice or three times by R₆,
each R₆ is independently selected from halogen, C₁-C₄alkyl, C₁-C₄alkoxy, C₁-C₄halogenalkyl, amino, aminoC₁-C₄alkyl, cyano, C₂-C₄alkynyl;
wherein C is neither a 2-aminopyridinyl nor a 6-aminopyridinyl residue.

2. The method according to claim 1 wherein
R₁ and R₂ are hydrogen; or
R₁ is methyl and R₂ is hydrogen.

3. The method according to claim 1 wherein
A is selected from phenyl, naphthyl, pyridinyl or quinolinyl.

4. The method according to claim 1 wherein
each R₃ independently is methyl, methoxy or
R₃ is pyridinyl, oxazolidinyl, pyrazolyl, pyrrolyl attached to A via —CH₂— or —SO₂—, wherein R₃ is optionally substituted with oxo.

5. (canceled)

6. The method according of claim 1 wherein
C is phenyl, pyridinyl, pyrrolopyridinyl, indolyl, indazolyl, isoquinolinyl or naphthyl.

7. The method according to claim 1 wherein
each R₆ is independently selected from chloro, fluoro, methyl, methoxy and trifluoromethyl.

8. A compound of formula (I) in free form or in pharmaceutically acceptable salt form which is selected from
N-((2,4-dimethylpyridin-3-yl)methyl)-1-((2-methylquinolin-6-yl)methyl)-1H-1,2,3-triazole-4-carboxamide;
N-((4-methoxy-2-methylpyridin-3-yl)methyl)-1-((2-methylquinolin-6-yl)methyl)-1H-1,2,3-triazole-4-carboxamide;
N-((6-hydroxy-2,4-dimethylpyridin-3-yl)methyl)-1-((2-methylquinolin-6-yl)methyl)-1H-1,2,3-triazole-4-carboxamide;
N-((2-hydroxy-3,5-dinnethylpyridin-4-yl)methyl)-1-((2-methylquinolin-6-yl)methyl)-1H-1,2,3-triazole-4-carboxamide;
N-((5-methyl-1H-pyrrolo[2,3-b]pyridin-3-yl)methyl)-1-((2-methylquinolin-6-yl)methyl)-1H-1,2,3-triazole-4-carboxamide;
N-((4-methyl-2-(trifluoromethyl)pyridin-3-yl)methyl)-1-((2-nnethylquinolin-6-yl)methyl)-1H-1,2,3-triazole-4-carboxamide;
1-(4-((1H-pyrazol-1-yl)methyl)benzyl)-N-(3-chlorobenzyl)-1H-pyrazole-4-carboxamide;
(1-(4-((1H-pyrazol-1-yl)methyl)benzyl)-N-(1-(3-chlorophenyl)ethyl)-1H-pyrazole-4-carboxamide;
N-((5-chloro-1H-indazol-3-yl)methyl)-1-(4-methoxybenzyl)-1H-pyrazole-4-carboxamide;
N-((5-chloro-1H-indol-3-yl)methyl)-1-((2-methylquinolin-6-yl)methyl)-1H-pyrazole-4-carboxamide;
N-((5-amino-3-methylpyrazin-2-yl)methyl)-1-((2-nnethylquinolin-6-yl)methyl)-1H-1,2,3-triazole-4-carboxamide;
N-(6-chloro-2-fluoro-3-methoxybenzyl)-1-((2-nnethylquinolin-6-yl)methyl)-1H-1,2,3-triazole-4-carboxamide;
N-((5-chloro-1H-indazol-3-yl)methyl)-1-(4-((2-oxopyridin-1(2H)-yl)methyl)benzyl)-1H-pyrazole-4-carboxamide;
N-((5-chloro-1H-indol-3-yl)methyl)-1-(4-((2-oxopyridin-1(2H)-yl)methyl)benzyl)-1H-pyrazole-4-carboxamide;
1-(4-((1H-pyrazol-1-yl)methyl)benzyl)-N-((5-chloro-1H-indazol-3-yl)methyl)-1H-pyrazole-4-carboxamide;
1-(4-((1H-pyrazol-1-yl)methyl)benzyl)-N-((5-chloro-1H-indol-3-yl)methyl)-1H-pyrazole-4-carboxamide;
N-((5-chloro-1H-indazol-3-yl)methyl)-1-(4-((2-oxooxazolidin-3-yl)methyl)benzyl)-1H-pyrazole-4-carboxamide;
N-((5-chloro-1H-indazol-3-yl)methyl)-1-((2-methylquinolin-6-yl)methyl)-1H-pyrazole-4-carboxamide;
1-(4-((1H-pyrrol-1-yl)sulfonyl)benzyl)-N-((5-chloro-1H-indazol-3-ypmethyl)-1H-pyrazole-4-carboxamide;
N-((5-chloro-1H-indol-3-yl)methyl)-1-(4-((2-oxooxazolidin-3-yl)methyl)benzyl)-1H-pyrazole-4-carboxamide;
1-(4-((1H-pyrrol-1-yl)sulfonyl)benzyl)-N-((5-chloro-1H-indol-3-yl)methyl)-1H-pyrazole-4-carboxamide;
N-((5-chloro-1H-indazol-3-yl)methyl)-1-(3,4-dimethoxybenzyl)-1H-pyrazole-4-carboxamide;
N-((5-chloro-1H-indazol-3-yl)methyl)-1-(3,5-dimethoxybenzyl)-1H-pyrazole-4-carboxamide;
N-((5-chloro-1H-indazol-3-yl)methyl)-1-(3-methoxybenzyl)-1H-pyrazole-4-carboxamide;
1-(4-((1H-pyrazol-1-yl)methyl)benzyl)-N-(3-chloro-2-fluorobenzyl)-1H-pyrazole-4-carboxamide; and
1-(4-((1H-pyrazol-1-yl)methyl)benzyl)-N-(1-(3-chloro-2-fluorophenyl)ethyl)-1H-pyrazole-4-carboxamide.

9. A compound which is selected from
N-(4-carbamimidoylbenzyl)-1-(naphthalen-2-ylmethyl)-1H-pyrazole-4-carboxamide hydrochloride; and
1-(4-((1H-pyrazol-1-yl)methyl)benzyl)-N-(4-carbamimidoylbenzyl)-1H-pyrazole-4-carboxamide hydrochloride.

10. A pharmaceutical composition comprising a therapeutically effective amount of a compound according to claim 1 in free form or in pharmaceutically acceptable salt form and one or more pharmaceutically acceptable carriers.

11. A combination comprising a therapeutically effective amount of the compound according to claim 1 in free form or in pharmaceutically acceptable salt form and one or more therapeutically active agents.

12-15. (canceled)

16. A pharmaceutical composition comprising a therapeutically effective amount of a compound according to claim 8 in free form or in pharmaceutically acceptable salt form and one or more pharmaceutically acceptable carriers.

17. A combination comprising a therapeutically effective amount of the compound according to claim 8 in free form or in pharmaceutically acceptable salt form and one or more therapeutically active agents.

18. A pharmaceutical composition comprising a therapeutically effective amount of a compound according to claim 9 in free form or in pharmaceutically acceptable salt form and one or more pharmaceutically acceptable carriers.

19. A combination comprising a therapeutically effective amount of the compound according to claim 9 in free form or in pharmaceutically acceptable salt form and one or more therapeutically active agents.
